(19) United States
(12) Patent Application Publication
(10) Pub. No.: US 2024/0352426 A1
(43) Pub. Date: Oct. 24, 2024

(54) Accelerated Protocol for the Differentiation of Podocytes from Human Pluripotent Stem Cells

(71) Applicants: Vanderbilt University, Nashville, TN (US); The United States as Represented by The Department of Veterans Affairs, Washington, DC (US)

(72) Inventors: Lauren E. WOODARD, Nashville, TN (US); Julie BEJOY, Nashville, TN (US)

(21) Appl. No.: 18/686,708

(22) PCT Filed: Aug. 25, 2022
(86) PCT No.: PCT/US2022/075447

§ 371 Date: Feb. 26, 2024

Related U.S. Application Data
(60) Provisional application No. 63/237,363, filed on Aug. 26, 2021.

(51) Int. Cl. C12N 5/071; A61K 35/545

(52) U.S. Cl. CPC C12N 5/0686; A61K 35/545; C12N 2500/38; C12N 2501/119; C12N 2501/155; C12N 2501/16; C12N 2501/165; C12N 2501/415; C12N 2501/727; C12N 2501/91; C12N 2506/45; C12N 2533/52

(57) ABSTRACT

Disclosed herein is a method for producing intermediate mesodermal cells from human pluripotent stem cells. Also disclosed is a method for producing nephron progenitor cells from human pluripotent stem cells. Also disclosed is a method for producing podocytes. Also disclosed is a method for treating a kidney disease in a subject, that involves administering to the subject a therapeutically effective amount of the produced intermediate mesodermal cells, nephron progenitor cells, and/or podocytes.

### CROSS-REFERENCE TO RELATED APPLICATIONS

This application claims benefit of U.S. Provisional Application No. 63/237,363, filed Aug. 26, 2021, which is hereby incorporated herein by reference in its entirety.

### SEQUENCE LISTING

This application contains a sequence listing filed in ST.26 format entitled “222230-2090 Sequence Listing” created on Aug. 12, 2022. The content of the sequence listing is incorporated herein in its entirety.

### BACKGROUND

Several kidney diseases including congenital nephrotic syndrome, Alport syndrome, and diabetic nephropathy are linked to podocyte dysfunction. Human podocytopathies may be modeled in either primary or immortalized podocyte cell lines. Human induced pluripotent stem cell (hiPSC)-derived podocytes are a new source of human podocytes, but the existing protocols have variable efficiency and expensive media components.

### SUMMARY

Disclosed herein is a feeder-free method for deriving functional, mature podocytes from pluripotent stem cells in only 12 days, saving time and money compared to other methods.

In particular, disclosed herein is a method for producing intermediate mesodermal cells from human pluripotent stem cells that involves (a) culturing the human pluripotent stem cells in a primitive streak induction medium comprising Activin A and/or Noggin and a glycogen synthase kinase 3 (GSK3) inhibitor and/or Wnt3a to produce posterior primitive streak cells; and (b) culturing the posterior primitive streak cells in an intermediate mesoderm induction medium comprising a GSK3 inhibitor to produce intermediate mesodermal cells. Also disclosed is a method for producing nephron progenitor cells from human pluripotent stem cells that involves (c) culturing the intermediate mesodermal cells of claim 1 in a nephron progenitor induction medium comprising fibroblast growth factor 9 (FGF9) and heparin to produce nephron progenitor cells. Also disclosed is a method for producing podocytes that involves (d) culturing the nephron progenitor cells of claim 2 in a podocyte induction medium comprising bone morphogenetic protein 7 (BMP7), vascular endothelial growth factor (VEGF), a GSK3 inhibitor, retinoic acid, and Activin A to produce podocytes.

In some embodiments, the GSK3 inhibitor is CHIR99021. In some embodiments, the primitive streak induction medium further comprises a Rho-associated, coiled-coil containing protein kinase (ROCK) inhibitor. In some embodiments, the ROCK inhibitor is Y-27632.

For example, in some embodiments, the primitive streak induction medium comprises 5-10 UM Y-27632, 10-100 ng/ml Activin A, and 1-3 UM CHIR99021. In some embodiments, the intermediate mesoderm induction medium comprises 6-10 μM CHIR99021. In some embodiments, the nephron progenitor induction medium comprise 50-200 ng/μl FGF9, and 1-10 μg/mL heparin. In some embodiments, the podocyte induction medium comprises 50-100 ng/ml BMP-7, 25-50 ng/ml VEGF, 1-3 UM CHIR99021, 0.1-1.0 UM all-trans retinoic acid, and 10-100 ng/ml Activin A.

In some embodiments, the human pluripotent stem cells, posterior primitive streak cells, intermediate mesodermal cells, nephron progenitor cells, or any combination thereof are cultured on a feeder free culture plate. For example, in some embodiments the culture plates are laminin coated.

In some embodiments, the method further involves assaying the posterior primitive streak cells produced in step (a) for MIXL1 expression. In some embodiments, the method further involves assaying the intermediate mesodermal cells produced in step (b) for paired gene box 8 (PAX8) and/or GATA3 expression. In some embodiments, the method further involves assaying the nephron progenitor cells produced in step (c) for SIX2 and/or CITED1 expression. In some embodiments, the method further involves assaying the podocytes produced in step (d) for synaptopodin (SYNPO), podocalyxin (PODXL), MAF BZIP transcription factor (MAFB), NPHS1 Adhesion Molecule (Nephrin), or any combination thereof.

In some embodiments, step (a) involves culturing the human pluripotent stem cells for 2-4 days to produce the posterior primitive streak cells. In some embodiments, step (b) involves culturing the posterior primitive streak cells for 2-3 days to produce the intermediate mesodermal cells. In some embodiments, step (c) involves culturing the intermediate mesodermal cells for 2-3 days to produce the nephron progenitor cells. In some embodiments, step (d) involves culturing the nephron progenitor cells for 5-7 days to produce the podocytes. Therefore, in some embodiments, the podocytes are produced from the human pluripotent stem cells within 12 days.

In some embodiments, the human pluripotent stem cells are induced pluripotent stem cells (iPSCs) or human embryonic stem cells. In some embodiments, the stem cells are genetically modified to model genetic kidney disease.

Also disclosed herein is a composition comprising intermediate mesodermal cells, nephron progenitor cells, and/or podocytes produced by the disclosed methods.

Also disclosed herein is a method that involves culturing the produced intermediate mesodermal cells, nephron progenitor cells, or podocytes in a cell culture medium.

Also disclosed herein is a method that involves using the cultured intermediate mesodermal cells, nephron progenitor cells, or mature podocytes to model podocytopathies in vitro.

Also disclosed herein is a method that involves using the cultured intermediate mesodermal cells, nephron progenitor cells to model diabetic nephropathy in vitro.

Also disclosed herein is a method that involves collecting the cell culture medium as a conditioned medium containing cytokines, exosomes, carbohydrates, lipids, proteins, and other molecules.

Also disclosed herein is a method that involves collecting extracellular matrix produced by the cultured intermediate mesodermal cells, nephron progenitor cells.

Also disclosed is a method for treating a kidney disease in a subject, that involves administering to the subject a therapeutically effective amount of the produced intermediate mesodermal cells, nephron progenitor cells, and/or podocytes. For example, in some embodiments, the kidney disease is diabetic nephropathy, chronic kidney disease, podocytopathies, nephrotoxic, or acute kidney injury. In some embodiments, the mature podocytes are administered by direct injection, culture in an implantable device, or implantation of a hydrogel.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a schematic diagram of the protocol for generating podocytes from hiPSCs. The protocol begins with differentiation of hiPSC to primitive streak using Activin A and CHIR, followed by induction of mesoderm with CHIR alone. Then, intermediate mesoderm was induced using FGF9. As final step to differentiate to podocytes, the intermediate cells were treated with combination of podocyte inducing factors including VEGF, Retinoic acid, BMP-7, CHIR and Activin A.

FIG. 2 shows morphological changes during each stage of differentiation. Brightfield images show differentiation of human iPS cells into primitive streak cells at day 3, intermediate mesoderm cells at day 6, nephron progenitors at day 8 and differentiated podocytes at day 13. The derived podocytes have a main body surrounded by elongated foot processes. The scale bar is 50 μM.

FIG. 3 contains brightfield and immunostaining images of the intermediate stages of podocyte differentiation: from human iPS cells to nephron progenitors. Undifferentiated human iPS cells stained positive for pluripotency marker Oct-4. iPSC differentiated into primitive streak cells at day 3 were positive for MIXL1. Day 6 intermediate mesoderm cells stained positive for PAX8. The nephron progenitors derived at day 8 stained positive for nephron progenitor markers CITED-1 and SIX2. Scale bar is 100 μM. iPSC: induced pluripotent stem cells; PS: primitive streak; IM: intermediate mesoderm; NP: nephron progenitors.

FIGS. 4A to 4D show characterization of podocyte cells derived from iPSCs. FIG. 4A shows lower and higher magnification brightfield images of the day 12 podocytes derived from the DYR0100 iPSC cell line. FIG. 4B shows flow cytometry analysis of the DYR0100-derived podocytes for podocyte markers MAFB and PODXL. FIG. 4C shows lower and higher magnification brightfield images of the day 12 podocytes derived from MAFB iPSC cell line. FIG. 4D shows flow cytometry analysis of the derived podocytes for expression of podocyte marker MAFB and the MAFB promoter-driven BFP stained with a FITC secondary antibody.

FIGS. 5A to 5C show immunofluorescence staining and electron micrograph images of human podocytes derived from iPSCs. FIG. 5A shows immunostaining of derived podocytes showing expression of podocyte lineage characterization markers WT1, MAFB, Synaptopodin, PODXL, Podocin, Nephrin, and F actin for cytoskeleton. Scale bar is 25 μM. FIG. 5B contains scanning electron microscopy images of iPSC podocytes showing cell bodies with cytoplasmic projections extending to adjacent cells (white arrows). FIG. 5C contains TEM images of the iPSC-derived podocytes showing tight junction-like structures between adjacent cell types (black arrows).

FIG. 6 shows functional validation of iPSC-derived podocytes. iPSC-derived podocytes were incubated with FITC-albumin for 1 h at 4° C. showing inhibition of endocytosis whereas 24 h at 37° C. showing endocytosis of the labeled albumin.

FIGS. 7A and 7B are brightfield images showing the iPS cells after accutase treatment. FIG. 7A shows prolonged accutase treatment showing over digestion resulting in single cells with more debris (white arrows) when resuspended. FIG. 7B shows cells treated for the optimal time showing clumps of cells (black arrows) after resuspension.

FIGS. 8A and 8B are brightfield images of a healthy vs unhealthy thaw of iPS cells. A desirable healthy thaw (FIG. 8A) has cells with a round morphology having clear edges whereas a thaw of unhealthy iPS cells (FIG. 8B) have mostly debris.

FIGS. 9A to 9C show undesirable plating densities of human iPS cells at day 0. FIG. 9A shows iPSC plated at too low density will fail to differentiate. Cells plated such low density will detach (arrows) and start to die following the primitive streak media treatment. FIG. 9B shows high density plating of iPSCs at day 0 will result in detachment of the cells (arrows) after the primitive streak media treatment (FIG. 9C).

FIGS. 10A and 10B show integration of iPSC-derived podocytes into mouse embryonic kidneys. FIG. 10A contains brightfield images of the recombinant organoids generated using iPSC-podocytes and E12.5 embryonic kidney at day 2 and day 7 after initiation of ex vivo co-culture. FIG. 10B contains higher (40×, i) and lower (20×, ii) magnification images showing the iPSC-derived podocytes (human-specific MAFB+) incorporated into mouse glomerular structures (mouse-specific NPHS1+) (n=2). Counterstaining with DAPI. Scale bar is 50 μm.

FIGS. 11A to 11D show modeling diabetic kidney disease by high glucose treatment of iPSC-derived podocytes. FIG. 11A contains brightfield images of cisplatin- or high glucose-treated podocytes showing damage to the junctions between cells (arrows). Scale bar represents 50 μm. FIG. 11B (i) shows reorganization of F-actin (phalloidin-red) along the cell periphery after the treatment with either cisplatin (5 μM) or high glucose (100 mM). Scale bar is 100 μm. FIG. 11B (ii) contains higher magnification images showing out-of-order and intertwined actin fibers in treated groups compared to control groups podocytes with paralleled fasciculate models (n=2). Scale bar is 30 μm. FIG. 11C shows viability analysis by MTT assay showing reduction in podocyte viability after treatment with glucose. FIG. 11D shows cytotoxicity analysis using LDH assay indicating increased toxicity within the podocytes treated with glucose. * Indicates p<0.05.

FIGS. 12A to 12C show modeling diabetic kidney disease by high glucose treatment of iPSC-derived podocytes. FIG. 12A shows Annexin V flow cytometry analysis showing 78.1% of apoptotic cells in the glucose-treated group compared to the 55.8% apoptotic cells in the control, untreated group. FIG. 12B shows immunostaining images with increased expression of cleaved caspase 3 in the glucose treated podocytes compared to control group. Scale bar is 100 μm. FIG. 12C are immunostaining images showing decreased expression of proliferation marker Ki67 in the glucose treated podocytes compared to control group. Scale bar is 100 μm.

FIGS. 13A and 13B show protocol overview and stages of iPSC-podocyte differentiation. FIG. 13A is a schematic of the podocyte differentiation protocol from iPSCs. FIG. 13B contains brightfield images of each stage of differentiation: primitive streak cells (Day 2), intermediate mesoderm (Day 5), nephron progenitors (Day 7), and podocytes (Day 12). The derived podocytes have a large cellular body with elongated foot processes. The scale bar is 100 μm.

FIGS. 14A to 14D show validation of each stage of podocyte differentiation. Immunostaining of each stage of differentiation showing pluripotency marker octamer-binding transcription factor 4 (OCT-4) at day-1 (FIG. 14A); primitive streak marker MIXL1 at day 2 (FIG. 14B); intermediate mesoderm marker PAX8 at day 5 (FIG. 14C); and nephron progenitor markers SIX2 and CITED-1 at day 7 (FIG. 14D). FIGS. 14E to 14H show corresponding flow cytometry analysis of the markers was plotted on the y-axis for OCT-4 (FIG. 14E), MIXL1 (FIG. 14F), PAX8 (FIG. 14G), and SIX2 (FIG. 14H) vs forward scatter (FSC) on the x-axis (n=2). Scale bar is 100 μm.

FIGS. 15A to 15E show characterization of human podocytes derived from iPSCs in 12 days. FIG. 15A shows immunostaining of iPSC podocytes showing podocyte lineage markers PODXL, P-CADHERIN, MAFB, SYNPO, and F actin for cytoskeleton. Scale bar is 100 μm. FIG. 15B shows pseudo colored scanning electron microscopy image of an iPSC-podocyte in blue showing large cell bodies with foot like projections and extracellular vesicles. Scale bar is 30 μm. FIG. 15C shows transmission electron microscopy image of podocytes showing tight junction-like structures between cells (black arrows). Scale bar is 400 nm. FIG. 15D shows flow cytometry histogram plots of the podocyte markers SYNPO and WT1 stained with a FITC and Texas Red secondary antibody (n=3). FIG. 15E shows quantitative analysis of podocyte markers such as PODXL, WT1, SYNPO and NEPHRIN throughout the differentiation process was analysed using (i) RT-PCR (ii) Western blot quantification with β-actin as control. * Indicates p<0.05.

FIGS. 16A to 16D show comparison of the protocol with existing protocols. FIG. 16A is a schematic of the podocyte differentiation protocols compared including key media components. FIG. 16B (i) is a comparison of the podocyte markers MAFB and PODXL expression using flow cytometry analysis (n=3). FIG. 16B (ii) shows quantification of (Bi). FIG. 16C shows immunostaining of iPSC podocytes with markers PODXL, NPHS1 in the compared protocols (n=3). Scale bar 100 μm. FIG. 16D shows Western blot analysis of the podocyte markers PODXL, SYNPO, NPHS1, and WT1 as well as loading control β-actin in the compared protocols (n=3).

FIGS. 17A and 18B show integration of iPSC-derived podocytes into mouse embryonic kidneys. FIG. 17A contains brightfield images of the recombinant organoids generated using iPSC-podocytes and E12.5 embryonic kidney at Day 2 and Day 7 after initiation of ex vivo co-culture. FIG. 17B shows higher (40×, i) and lower (20×, ii) magnification images showing the iPSC-derived podocytes (human-specific MAFB+) incorporated into mouse glomerular structures (mouse-specific NPHS1+) (n=2). Counterstaining with DAPI. Scale bar is 50 μm.

FIGS. 18A to 18D show modeling diabetic kidney disease by high glucose treatment of iPSC-derived podocytes. FIG. 18A contains brightfield image of cisplatin- or high glucose-treated podocytes showing damage to the junctions between cells (arrows). Scale bar represents 50 μm. FIG. 18B (i) shows reorganization of F-actin (phalloidin-red) along the cell periphery after the treatment with either cisplatin (5 μM) or high glucose (100 mM). Scale bar is 100 μm. FIG. 18B (ii) contains higher magnification images showing out-of-order and intertwined actin fibers in treated groups compared to control groups podocytes with paralleled fasciculate models (n=2). Scale bar is 30 μm. FIG. 18C shows viability analysis by MTT assay showing reduction in podocyte viability after treatment with glucose. FIG. 18D shows cytotoxicity analysis using LDH assay indicating increased toxicity within the podocytes treated with glucose. * Indicates p<0.05.

FIGS. 19A and 19B show characterization by flow cytometry of pluripotency and primitive streak markers at each stage of hiPSC-podocyte differentiation. FIG. 19A shows flow cytometry analysis of pluripotency marker OCT-4 at each stage of differentiation. First panel is identical to FIG. 2E as the samples are from the same experiment. FIG. 19B shows flow cytometry analysis of primitive streak marker MIXL1 expression at each stage of differentiation. Markers are plotted on the y-axis; forward scatter (FSC) is plotted on the x-axis.

FIG. 20 show characterization of podocyte cells derived from three iPSC cell lines. Lower (i) and higher (ii) magnification brightfield images of the day 12 podocytes derived from the (A) DYR0100 iPSC cell line; (B) MAFB:mTagBFP2/GATA3:mCherry iPSC cell line; and (C) LRP2BFP:mTagBFP2 cell line.

FIG. 21 shows functional validation of iPSC-derived podocytes. iPSC-derived podocytes were incubated with FITC-albumin either for 1 h at 4° C. or 24 h at 37° C. showing endocytosis of the labeled albumin only at 37° C. Scale bar is 100 μm.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

Provided herein are compositions, systems, kits, and methods for generating human podocyte cells. In certain embodiments, the podocytes generated by the methods and compositions here are used in different applications where podocytes are required, including, as an in vitro model for a kidney/glomerular disorder, therapeutic applications (e.g., tissue regeneration and/or repair or transplantation), drug discovery and/or developments, and/or tissue engineering. In certain embodiments, the methods comprises culturing in a cell or tissue culture device the isolated population of podocytes described herein.

In certain embodiments, provided herein are synthetic tissue scaffold comprising a cell-compatible biopolymer and an isolated population of podocytes distributed therein, wherein the isolated population of podocytes is produced by the methods and compositions described herein. Examples of a cell-compatible biopolymer include, but are not limited to, silk fibroin, polyethylene oxide (PEO), polyethylene glycol (PEG), fibronectin, keratin, polyaspartic acid, polylysine, chitin, hyaluronic acid, pectin, polycaprolactone, polylactic acid, polyglycolic acid, polyhydroxyalkanoates, dextrans, polyanhydrides, polymer, PLA-PGA, polyanhydride, polyorthoester, polycaprolactone, polyfumarate, collagen, chitosan, alginate, hyaluronic acid, and/or other biocompatible polymers. Also provided herein is a biological ink comprising the isolated population of podocytes described herein mixed with a viscous extracellular matrix for use in a 3-D printer. In some embodiments, the isolated population of podocytes described herein can be mixed with a viscous gelatin to form a biological ink of podocytes. The resulting biological ink can be fed into a 3-D printer, which is programmed to arrange different cell types, along with other materials, into a precise three-dimensional shape.

In some embodiments where normal, healthy podocytes are used, the podocytes can be contacted with an agent that induces the podocytes to acquire at least one phenotypic characteristic associated with a kidney and/or glomerular disorder, thereby modeling a kidney and/or glomerular disorder in vitro. In some embodiments, doxorubicin (Adriamycin) can be introduced to induce podocytes injury to model a kidney or glomerulus-specific condition in vitro.

In certain embodiments, a method of screening for an agent to reduce at least one phenotypic characteristic of podocytes associated with a kidney and/or glomerular disorder is provided herein. The method comprises (a) culturing the isolated population of podocytes described herein that display at least one phenotypic characteristic associated with the kidney and/or glomerular disorder; (b) contacting the podocytes with a library of candidate agents; and (c) detecting response of the podocytes to the candidate agents to identify an agent based on detection of the presence of a reduction in the phenotypic characteristic of the podocytes associated with the kidney and/or glomerular disorder. The candidate agents can be selected from the group consisting of, for example, proteins, peptides, nucleic acids (e.g., but not limited to, siRNA, anti-miRs, antisense oligonucleotides, and ribozymes), small molecules, and a combination of two or more thereof. The effects of the candidate agents on the podocytes can be determined by measuring response of the cells and comparing the measured response with podocytes that are not contacted with the candidate agents.

In some embodiments, the podocytes generated by the differentiation methods described herein and/or synthetic tissue scaffolds described herein can be used for kidney regeneration or as cell-based therapeutics for treatment of a kidney and/or glomerular disorder (including, e.g., podocyte injury, proteinuria, glomerulosclerosis, diabetic nephropathy, chemotherapy-related nephrotoxicity or combinations thereof). Thus, methods of treating a kidney and/or glomerular disorder are also provided herein. In one embodiment, the method comprises transplanting to a subject in need thereof (e.g., suffering from a kidney and/or glomerular disorder) an isolated population of podocytes generated by the methods herein and/or a synthetic tissue scaffold described herein. In some embodiments, the podocytes and/or the synthetic tissue scaffold can be transplanted at or in close proximity to a pre-determined location of a kidney of the subject. For example, the podocytes and/or the synthetic tissue scaffold can be transplanted at or in close proximity to a damaged area of a kidney of the subject. The transplanted podocytes can migrate and localize into at least one or more glomerular capillary structure of the kidney tissue, thereby facilitate regeneration and/or repair of the kidney tissue. In some embodiments, the podocytes can be encapsulated within permeable matrices prior to implantation. Encapsulation provides a barrier to the host's immune system and inhibits graft rejection and inflammation. Several methods of cell encapsulation can be employed. In some instances, podocytes can be individually encapsulated. In other instances, many cells can be encapsulated within the same matrix.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: Accelerated Protocol for the Differentiation of Podocytes from Human Pluripotent Stem Cells

### Development of the Protocol

Podocytes are highly differentiated cells with long foot processes that wrap around the renal capillaries to provide glomerular filtration. Most glomerular disorders are associated with phenotype alterations in proliferating podocytes whose malfunction leads to proteinuria (Barisoni, L., et al. J Am Soc Nephrol. 1999 10:51-61). Animal models have been extensively used in research to better understand the genesis and progression of podocytopathies as well as to find possible drug targets and treatments (Pippin, J. W., et al. Am J Physiol Renal Physiol. 2009 296: F213-F229). Despite this, animal models do not always replicate human analogues or disease characteristics, necessitating the development of robust and reproducible methods for the in vitro culture of human podocytes. The reprogramming of human induced pluripotent stem cells (hiPSCs) from adult human cells has opened up new techniques to generate various cell types in vitro through directed differentiation (Takahashi, K., et al. Cell 2007 131:861-872). Utilizing a strong basis of knowledge gained from studies of embryonic kidney development, multiple approaches for generating podocytes from hiPSCs have been developed (Musah, S., et al. Nat Biomed Eng. 2017a 1:0069; Rauch, C., et al. PloS one 2018 13, e0203869-e0203869; Song, B., et al. PloS one 2012 7: e46453).

The embryonic development of the kidney starts with the generation of primitive streak. The primitive streak is an elongating groove-like structure that forms in the posterior region of the blastula (Downs, K. M. BioEssays 2009 31:892-902). The cells then migrate from the late primitive streak to form the mesoderm which consists of paraxial, intermediate, and lateral plate cells. The anterior region of the primitive streak forms the paraxial mesoderm whereas the posterior region forms the lateral plate mesoderm (Wilson, V., et al. Mech Dev. 1996 55:79-89). The intermediate mesoderm (IM) lies between the paraxial and lateral plate mesoderm. IM cells are the source of kidney progenitor populations. The two major progenitors present in the kidney are the ureteric epithelium that develops into the collecting duct/ureter (Mendelsohn, C. Organogenesis 2009 5:306-314) and the metanephric mesenchyme that differentiates into the remaining structures of the nephron including the glomerular podocytes (Kobayashi, A., et al. Cell Stem Cell 2008 3:169-181). Within the intermediate mesoderm, the anterior region differentiates to the ureteric epithelium and the posterior region differentiates into the nephron progenitor cells (NPCs) (Taguchi, A., et al. Cell Stem Cell 2014 14:53-67). Since podocytes arise from the cap mesenchyme, metanephric mesenchyme induction from iPSCs is required for efficient podocyte production.

The type of extracellular matrix (ECM) is critical for the support and adhesion of iPSCs and also for the efficient differentiation to podocytes. Interaction between cells and ECM is mediated via integrins, which are the transmembrane receptors consisting of α- and β-subunits. Integrins β1 and αvβ5 are highly expressed in both human iPSC and in a human podocyte cell line (Musah, S., et al. Nat Biomed Eng. 2017a 1:0069). β1 integrins are essential for podocyte function in vivo (Kanasaki, et al. Dev Biol. 2008 313:584-593; Pozzi, A., et al. Dev Biol. 2008 316:288-301.). Laminin 5-11 (LN-511), which consists of α5, β1, and γ1 chains, has been reported to promote greater adhesion of hESCs and hiPSCs than matrigels and other matrices (Rodin, S., et al. Nat Biotechnol. 2010 28:611-615). Taking each of these factors into consideration, the protocol described below was developed. This protocol produces podocytes from human iPSC with ˜70% efficiency, comparable to most of the existing protocols, in a short time period.

### Primitive Streak Induction

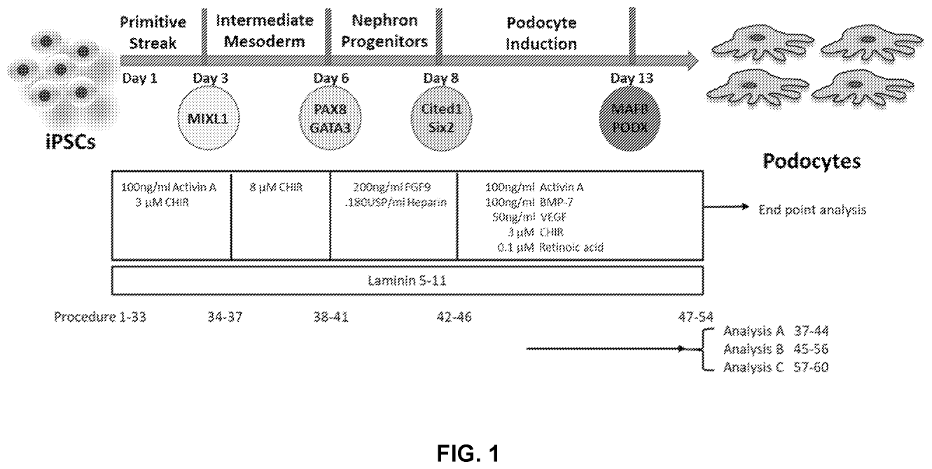

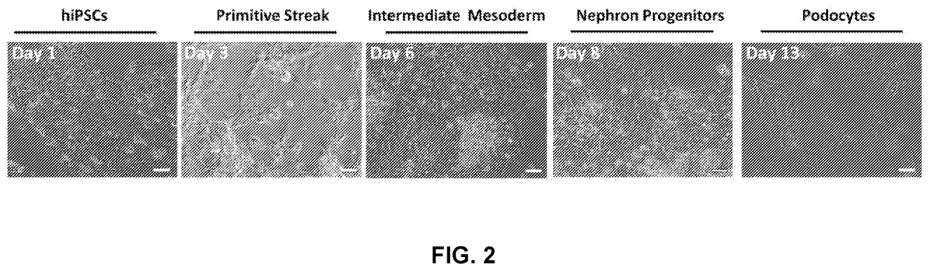

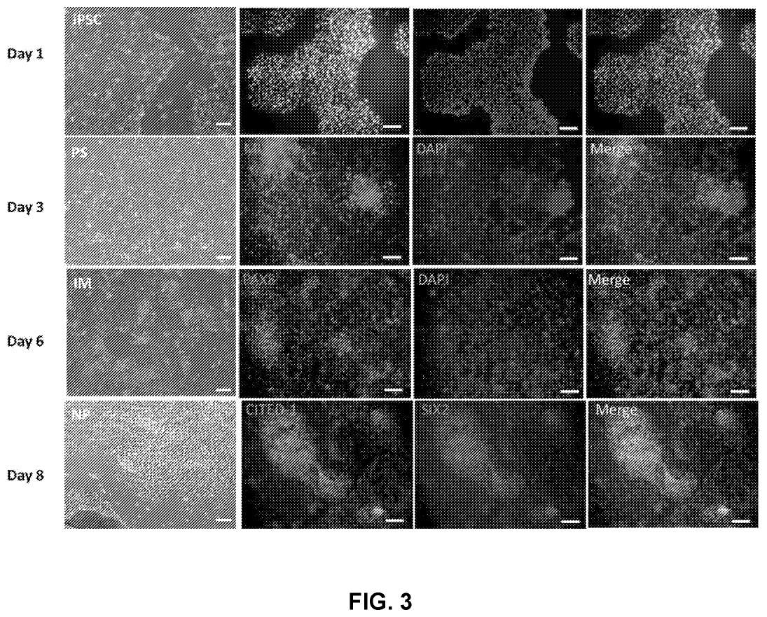

Most accelerated protocols for podocyte differentiation either employ the embryoid body (EB) formation method or use media containing serum. The use of EBs in other protocols gives rise to cell-to-cell heterogeneity, while serum-containing media results in inconsistent results due to batch effects. The existing serum-free protocols require longer culture time which is usually undesirable. Therefore, the goal was to generate an accelerated protocol containing serum-free media to generate podocytes (FIG. 1). The first step of differentiation is the induction of the late primitive streak. Wnt and TGF-β/nodal/activin signaling are simultaneously required for the generation of the Brachyury+ primitive streak population (Gadue, P., et al. Proc Natl Acad Sci USA. 2006 103:16806-16811; Tam, P. P., et al. Nat Rev Genet. 2 2007 8:368-381). These signals can efficiently generate posterior primitive streak from hPSCs (Morizane, R., et al. Nature protocols 2017 12:195-207; Musah, S., et al. Nat Biomed Eng. 2017a 1:0069; Taguchi, A., et al. Cell Stem Cell 2014 14:53-67; Takasato, M., et al. Nat Cell Biol. 2014 16:118-126; Takasato, M., et al. Nature 2015 526:564-568). The cells are cultured under monolayer conditions to achieve more precise anterior-posterior cell fate. Activin A has also been reported to differentiate hPSCs to primitive streak (Takasato, M., et al. Nat Cell Biol. 2014 16:118-126). Therefore, a combination of a low dose of canonical WNT signaling and Activin A was used for the first 2 days to generate primitive streak. These cells exhibited changed morphology (FIG. 2) and stained positive for the primitive streak marker MIXL1 (FIG. 3).

### Intermediate Mesoderm Induction

The second stage of podocyte differentiation is the differentiation of primitive streak cells into the intermediate mesoderm. ESCs and iPSCs require canonical Wnt signaling activation for posterior primitive streak and intermediate mesoderm induction (Kreuser, U., et al. Front Cell Dev Biol. 2020 8; Lindsley, R. C., et al. Development 2006 133:3787-3796; Mae, S.-I., et al. Nat Commun. 2013 4:1367). Alternative protocols used a combination of BMP7 and Wnt to derive intermediate mesoderm from iPSCs (Musah, S., et al. Nat Biomed Eng 2017b 1). Therefore, primitive streak cells were treated with a high dose of the Wnt activator CHIR for three days to induce intermediate mesoderm formation. The cells began to proliferate and started to form thick layers at this stage of differentiation (FIG. 2). These cells expressed the intermediate mesoderm cell marker paired box gene 8 protein (PAX8) (FIG. 3), which is a critical regulator of the nephric lineage specification (Bouchard, M., et al. Genes Dev. 2002 16 (22): 2958-70).

### Nephron Progenitor Induction

Because the primitive streak differentiates spontaneously into the lateral plate mesoderm, exogenous factors to direct differentiation to the medial plate are necessary. At this stage, cells include progenitors of the ureteric epithelium, metanephric mesenchyme, renal interstitium, and endothelium (Mugford, J. W., et al. Dev Biol. 2008 324:88-98). The metanephric mesenchyme is derived from the posterior intermediate mesoderm (PIM) so the next step is to induce nephron progenitors from these cells. Most existing protocols used a combination of three or four factors to induce nephron progenitor formation. Morphogens such as BMP4 and FGF9 induce the medial-lateral patterning of the trunk mesoderm. BMP4 is expressed in the lateral plate mesoderm (James, R. G., et al. Dev Biol. 2005 288:113-125) whereas FGF9 is expressed in the intermediate mesoderm (Colvin, J. S., et al. Dev Dyn. 1999 216:72-88). When starting from iPSCs, FGF9 alone is sufficient to specify the intermediate mesoderm and generate nephron progenitors (Ciampi, O., et al. Stem Cell Res 2016 17:130-139; Low, J. H., et al. Cell Stem Cell 2019 25:373-387.e379). FGF9 can also maintain nephron progenitors in vitro (Barak, H., et al. Dev Cell. 2012 22:1191-1207). Therefore, in order to simplify the procedure FGF9 alone was used to generate nephron progenitors (FIG. 1). Heparin causes oligomerization of FGFs and supports their binding to FGF receptors, resulting in their activation (Spivak-Kroizman, T., et al. Cell 1994 79:1015-1024). Therefore, heparin is used together with FGF9 in many existing kidney differentiation protocols (Morizane, R., et al. Nat Biotechnol 2015 33:1193-1200; Takasato, M., et al. Nature 2015 526:564-568). Compared to other protocols, the nephron progenitor formation using FGF9 required a short window of only 2 days. FGF9 was added together with heparin until day 7 to induce the generation of nephron progenitors. The cells continued to proliferate and thick cobblestone-like morphology was seen in the culture (FIG. 2), as previously reported (Pleniceanu, O., et al. Pediatr Res. 2018 83:267-274). The cells derived stained positive for the nephron progenitor cell markers SIX2 and CITED1 at day 7 (FIG. 3).

### Podocyte Induction

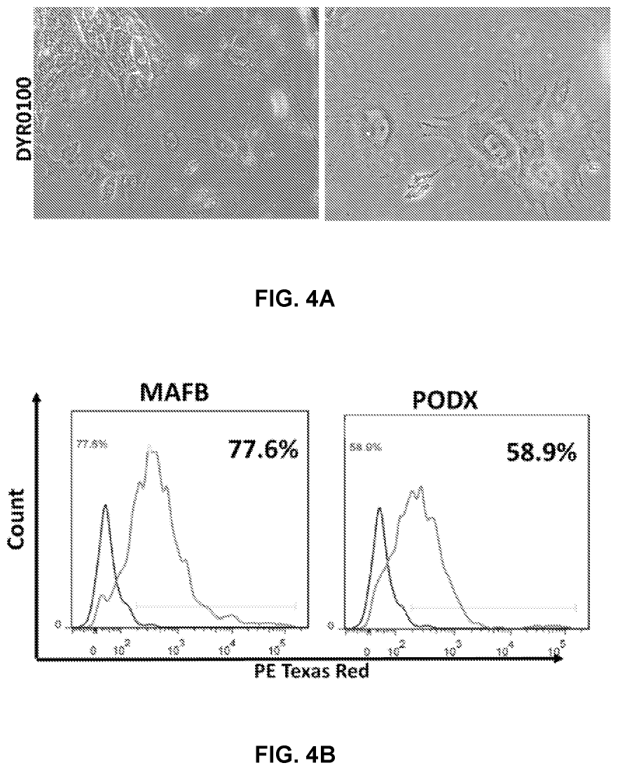

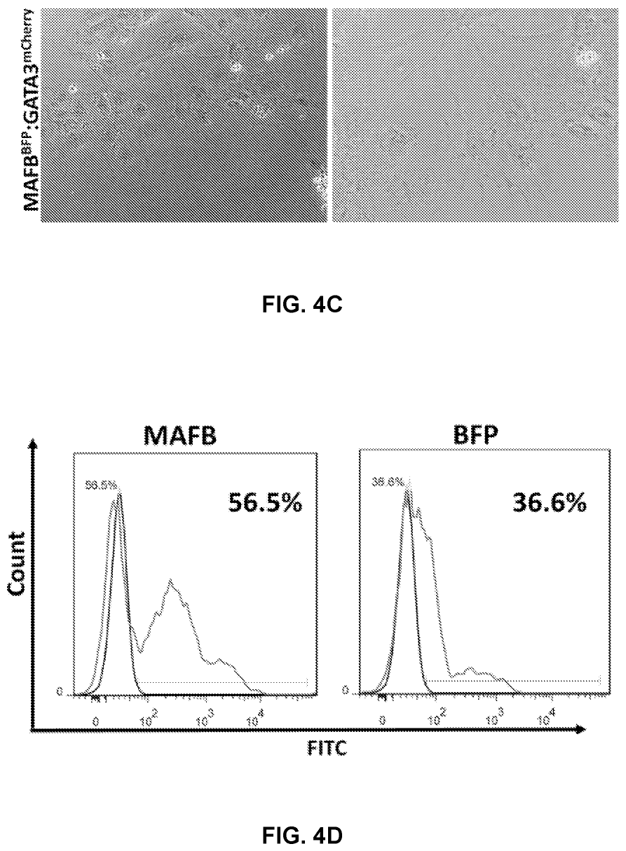

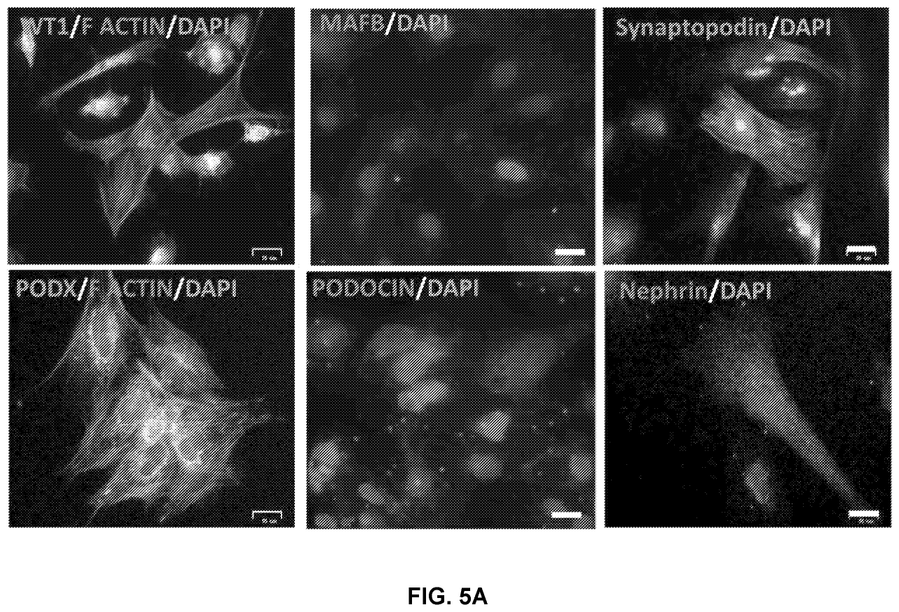

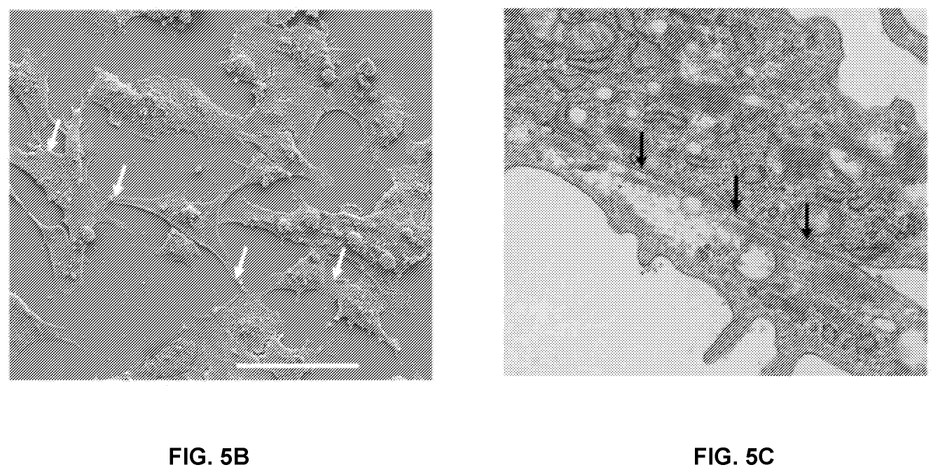

Growth factor cocktails including retinoic acid, BMP7, vascular endothelial growth factor (VEGF), and activin A are effective for generating podocytes from iPSCs (Rauch, C., et al. PloS one 2018 13, e0203869-e0203869). Retinoic acid (RA) encourages the differentiation of podocytes (Dai, Y., et al. Kidney Int. 2017 92:1444-1457; Mallipattu, S. K., et al. Front Med (Lausanne) 2015 2:16-16; Vaughan, M. R., et al. Kidney Int. 2005 68:133-144), whereas BMP7 is both a podocyte differentiation and survival factor (Mitu, G. M., et al. Am J Physiol Renal Physiol. 2007 293: F1641-1648). VEGF is crucial for endothelial cell development. But VEGF also supports the survival of podocytes both in vitro and in vivo (Harper, S. J., et al. Clin Sci (Lond). 2001 101 (4): 439-46). To induce the differentiation of podocytes from the nephron progenitors, a cocktail of these factors was used. The nephron progenitors were dissociated and plated them onto laminin 5-11 coated plates to support the adhesion and maturity of podocytes. The recombinant laminin-511 E8 fragment used in the protocol is composed of the C-terminal regions of the alpha, beta, and gamma chains which bind to integrin a631, located on the cell surface. The matrix has been reported to be essential for integrin-receptor-mediated glomerular basement membrane (GBM) signaling (Maier, J. I., et al. Cell Reports 2021 34:108883; Suleiman, H., et al. Elife. 2013 2: e01149) and has been used for podocyte differentiation (Musah, S., et al. Nat Biomed Eng 2017b 1). Most existing protocols require 6-10 days of differentiation to derive podocytes from nephron progenitors (Ciampi, O., et al. Stem Cell Res 2016 17:130-139; Qian, T., et al. Sci Adv. 2017 3: e1701679; Rauch, C., et al. PloS one 2018 13, e0203869-e0203869). However, this protocol has reduced time required for the generation of podocytes from nephron progenitors to 5 days. The cell morphology changed into a large arborized structure with a large cell body (FIG. 4A,4C). Scanning electron microscopy (SEM) showed that the cells exhibit prominent primary thin processes (FIG. 5B) and cells visualized with transmission electron microscopy (TEM) displayed tight junctions (FIG. 5C). The resulting cells expressed podocyte lineage specification markers including: Synaptopodin (SYNPO), Podocalyxin (PODXL), MAF BZIP transcription factor (MAFB), and NPHS1 Adhesion Molecule (Nephrin) (FIG. 5A).

### Materials

Table 1 is a list of reagents or resources used in this protocol.

TABLE 1
Resources

| | | |
|---|---|---|
| Reagent or Resource | Source | Identifier |

Antibodies

| | | |
|---|---|---|
| Rat Monoclonal anti-Oct-4 | R& D systems | Cat# MAB1759SP |
| Rabbit Polyclonal anti-MIXL1 | Proteintech | Cat#22772-1-AP |
| Rabbit Polyclonal anti-PAX8 | Proteintech | Cat#10336-1-AP |
| Goat Polyclonal anti-GATA3 | R& D systems/ | Cat#AF2605 |
| Mouse Monoclonal anti-Cited-1 | Fisher Scientific | Cat#89-335-107 |
| Rabbit Polyclonal anti-SIX2 | Proteintech | Cat#11562-1-AP |
| Rabbit Polyclonal anti-MAFB | Abcam | Cat#ab223744 |
| Rabbit Polyclonal anti-PODX | Proteintech | Cat#18150-1-AP |
| Rabbit Polyclonal anti-WT1 | Proteintech | Cat#12609-1-AP |
| Sheep Polyclonal anti-NEPHRIN | R& D systems | Cat#AF4269 |
| Rabbit Polyclonal anti- | Abcam | Cat#ab224491 |
| Synaptopodin | | |
| Alexa 488, goat anti-mouse IgGa | Life Technologies | Cat#A-21131 |
| Alexa 488, goat anti-rat IgG | Life Technologies | Cat#A-11006 |
| Alexa 488, donkey anti-goat IgG | Life Technologies | Cat#A-11055 |
| Alexa 594, goat anti-rabbit IgG | Life Technologies | Cat#A-11037 |
| Alexa 594, donkey anti-sheep IgG | Life Technologies | Cat#A-11016 |

Chemicals, peptides, and recombinant proteins

| | | |
|---|---|---|
| mTeSR medium | STEMCELL | Cat#05825 |
| | Technologies | |
| Y27632 ROCK inhibitor | STEMCELL | Cat#72304 |
| | Technologies | |
| Accutase | Fisher Scientific | Cat#A1110501 |
| DMEM/F12 with GlutaMAX | Thermo Fisher | Cat#10565042 |
| supplement | Scientific | |
| B27 serum-free supplement | Thermo Fisher | Cat#17504044 |
| | Scientific | |
| Laminin-511 (BG iMatrix-511) | PeproTech | Cat#RL511 |
| Human Activin A | PeproTech | Cat#120-14P-10 ug |
| CHIR99021 | Reagents Direct | Cat#27-H76 |
| Human BMP7 | Fisher Scientific | Cat#354-BP |
| Human VEGF | Millipore Sigma | Cat#V7259 |
| All-trans retinoic acid | Stem Cell | Cat#72262 |
| | Technologies | |
| Fetal bovine serum | Life Technologies | Cat#26140079 |
| Penicillin-Streptomycin | Mediatech/CellGro- | Cat#30002CI |
| | Corning | |
| BSA | Sigma Aldrich | Cat#A7030-50 g |
| DMSO | Sigma Aldrich | Cat#D2650 |
| Phosphate-buffered saline | Mediatech/CellGro- | Cat#21-040CV |
| (PBS), 1X | Corning | |
| w/o Calcium & Magnesium | | |
| Phosphate-buffered saline | Mediatech/CellGro- | Cat#MT46013CM |
| (PBS), 10X | Corning | |
| w/o Calcium & Magnesium | | |
| Cell culture grade water | Millipore Sigma | Cat#W3500-500 ML |
| Deionized water | n/a | n/a |
| FGF9 | PeproTech | Cat#100-23-50 ug |
| Heparin | Sigma Aldrich | Cat#H3149-100 KU |
| Geltrex ™, LDEV-Free | Fisher Scientific | Cat#A1413202 |
| Matrigel | Fisher Scientific | Cat#CB-40230A |
| Trypsin EDTA, 0.05% | Thermo Fisher | Cat#25-300-054 |
| | Scientific | |
| Paraformaldehyde | Fisher Scientific | Cat#50-980-487 |
| Triton X-100 (TritonX) | Sigma-Aldrich | Cat#T9284 |
| CryoStor CS-10 | STEMCELL | Cat#07959 |
| CRYOPRESERVATION MEDIUM | Technologies | |
| DAPI (6-Diamidino-2-Phenylindole | Sigma-Aldrich | Cat#D9542-5 MG |
| Dihydrochloride) | | |
| Methanol | Sigma Aldrich | Cat#D2650 |
| FITC albumin | Thermo Fisher | Cat#A23015 |
| | Scientific | |
| F-actin (Phalloidin 594) | Molecular grade | Cat#A12381 |
| | Probe | |

Experimental models: Cell lines

| | | |
|---|---|---|
| Human iPSC | ATCC | Cat#DYR0100 |

Software and algorithms

| | | |
|---|---|---|
| Image J | Schneider et al., | https://imagej.nih.gov/ij/ |
| | 2012 | |
| Flow Jo | BD Biosciences | https://www.flowjo.com/ |

Others

| | | |
|---|---|---|
| Biological safety cabinet | NUAIRE, Class II | n/a |
| | Type A2 | |
| Water bath, 37° C. | Fisher Scientific, | n/a |
| | Isotemp 2332 | |
| Benchtop centrifuge | Eppendrof, | n/a |
| | Centrifuge 5424 | |
| CO₂ incubators | NUAIRE | Cat#NU5500 |
| Conical tubes (15, 50 mL) | VWR | Cat#490001-621 |
| | | Cat#490001-627 |
| Cryogenic Storage Vials | Greiner Bio-One | Cat#122263 |
| Cryovial Freezer | BioExpress | Cat#BCS-405O |
| Inverted contrasting tissue culture | Zeiss, Primovert | n/a |
| microscope | | |
| Laser confocal microscope | Nikon, Spinning | n/a |
| | Disk | |
| Flow Cytometer | BD, BD | n/a |
| | LSRFortessa | |
| Media storage bottle | Fisher Scientific | Cat#07-201-600 |
| Media storage bottle | Fisher Scientific | Cat#431531 |
| Pipette Controller | Fisher Scientific | Cat#14-389-136TU |
| Pipette Set | Fisherbrand (P2, | Cat#FBE00002 |
| | P10, P200 and | Cat#FBE00010 |
| | P1000 Elite) | Cat#FBE00200 |
| | | Cat#FBE10000 |
| Serological pipettes | VWR | Cat#82050-482 |
| | | Cat#82050-478 |
| Steriflip 0.22 μm filter unit | Fisher Scientific | Cat#SCGP00525 |
| Sterile filter pipette tips(10, 20 mL) | VWR | Cat#76322-132 |
| | | Cat#76322-134 |
| Sterile filter pipette tips (200, wide | Fisher Scientific | Cat#NC1915695, |
| bore 200, 1000 μl) | | Cat#14222730, |
| | | Cat#NC1915694 |
| Sterile microcentrifuge tube | Fisher Scientific | Cat#02-707-352 |
| Swing-out rotor centrifuge | Thermo IEC, | n/a |
| | Centra CL3R | |
| Tissue culture-treated plates (12 | Fisher Scientific | Cat#08-772-29 |
| wells, 6 well) | | Cat#07-200-83 |

### Reagent Setup

### Geltrex

First, thaw the Geltrex by placing it on ice. To prepare 1% Geltrex, add 400 μl of Geltrex into a 50 mL tube containing 40 mL of chilled DMEM/F-12. Make 1 ml aliquots and store them at −20° C. until ready to use. Add 1 mL of aliquot to each well of the 6 well plates and keep it at 37° C. for at least 1 h to allow Geltrex to coat the surface. Use the coated plate within 48 h, storing at 4° C. Handle Geltrex on ice as it solidifies when warmed over 16° C. The pipette tips used for dilution should be pre-chilled by keeping the pipette tips at −20° C. for 30 min before use. Coated plates must be used within 48 hours. Plates kept longer will dry out and the cells will not attach to the plates. Do not use dry plates.

### Matrigel

First, thaw Matrigel by placing it on ice. To prepare 1:30 diltution of Matrigel, add 1 ml of Matrigel into a 50 ml tube containing 29 mL of chilled DMEM/F-12. Make 1 ml aliquots and store them at −20° C. until ready to use. Add the 1 mL aliquot to each well of a 6 well plate and keep it at 37° C. for at least 1 h to allow the Matrigel to coat the surface of the well. Use the coated plate within 48 h, storing at 4° C. Handle Matrigel on ice as it solidifies when warmed over 16° C. The pipette tips used for dilution should be pre-chilled by keeping the pipette tips at −20° C. for 30 min before use. Coated plates must be used within 48 hours. Plates kept longer will dry out and the cells will not attach to the plates. Do not use dry plates.

mTeSR Medium

Combine 400 mL of mTeSR basal medium, 100 mL of 5× supplement, and 5 mL of 100× Penicillin/Streptomycin to make the stock media. Aliquot this prepared media into 50 mL conical tubes and store at −20° C. for up to six months. Thawed aliquots may be kept in the refrigerator at 4° C. and used within two weeks.

### Y27632 (10 mM)

Resuspend 1 mg of Y27632 in 312 μL of PBS (pH 7.2) or cell culture grade water to make a 10 mM stock. Prepare 50 μl aliquots and store at −20° C. for up to six months. Avoid freeze-thaw cycles.

Human Activin a (100 μg/mL)

Centrifuge the tube briefly before opening. Reconstitute to 100 μg of Activin A in 1 mL of sterile cell culture grade water containing 0.1% BSA. Prepare 20 μl aliquots and store at −20° C. for six months. Avoid freeze-thaw cycles.

### CHIR99021 (10 mM)

Centrifuge the tube briefly before opening. Reconstitute the 2 mg vial by adding 430 μl of DMSO to make a 10 mM stock. Prepare 10 μl aliquots and store at −20° C. or −80° C. for up to 12 months. Avoid repeated freeze-thaw cycles. The reconstituted vial can be stored in 2° C. to 8° C. for a week.

FGF9 (200 μg/mL)

Centrifuge the tube briefly before opening. Reconstitute to 50 μg in 250 μl of sterile cell culture grade water containing 0.1% BSA. Prepare 10 μl aliquots and store at −20° C. or −80° C. for up to three months. Avoid freeze-thaw cycles. The reconstituted vial can be stored in 2° C. to 8° C. for one week.

### Heparin Solution (180 USP)

Reconstitute in 180 USP of heparin in 1 ml sterile cell culture grade water and filter sterilize it through a polyethersulfone (PES) 0.22 μm syringe-driven filter unit to derive the 180 USP/mL heparin stock. Prepare 10 μl aliquots and store. Stocks are stable at 2° C. to 8° C. for at least 12 months.

Human BMP7 (100 μg/mL)

Prepare the reconstitution solution by making a solution of 4 mM HCl containing 0.1% (wt/v) BSA and sterilize using a syringe-driven filter unit. To make the 100 μg/mL stock of BMP7, add 100 μl of reconstitution solution to 10 μg of BMP7. Prepare 10 μl aliquots and store at −20° C. or −80° C. for up to three months. Avoid freeze-thaw cycles. The reconstituted vial can be stored in 2° C. to 8° C. for one month.

Human VEGF (50 μg/mL)

Reconstitute 50 μg in 1 ml sterile cell culture grade water to make 50 μg/mL stocks. Prepare 10 μl aliquots and store at −20° C. or −80° C. for up to 12 months. Avoid repeated freeze-thaw cycles. The reconstituted vial can be stored at 2° C. to 8° C. for one week.

### All-Trans Retinoic Acid

To prepare a 100 UM stock solution, resuspend 100 μg of all-trans retinoic acid in 3.33 mL of sterile DMSO. Prepare the stock solution fresh before use, or aliquot into working volumes and store at −20° C. Avoid repeated freeze-thaw cycles.

### Paraformaldehyde (PFA) (4%)

Inside a fume hood, carefully break the glass vial containing 10 mL of a 16% PFA solution. Add the 10 mL of 16% PFA to a 50 ml conical vial. Add 4 mL of 10× PBS and 26 mL of deionized water to make 40 mL of a 4% (wt/vol) paraformaldehyde in PBS solution. The 4% PFA solution can be stored at room temperature for 1-2 weeks or at 4° C. for 3 weeks. For long term storage, aliquot and keep at −20° C. for up to a year. Paraformaldehyde is toxic and must be handled inside a fume hood. Personnel should wear the appropriate personal protective equipment such as gloves, a lab coat, a face mask, and goggles.

### 1× Phosphate-Buffered Saline, 0.1% Tween 20 Detergent (PBS-BT)

To prepare PBS-BT, add 6 g BSA to 20 mL 10×PBS. To this, add 2 mL of a 10% solution of Tween 20 and 2 mL of a 2% solution of sodium azide. Add sterile deionized water to bring the total volume of the solution to 200 mL. Filter sterilize and store at 4° C. Sodium azide is a toxic preservative and must be handled inside a fume hood. Personnel should wear the appropriate personal protective equipment such as gloves, a lab coat, a face mask, and goggles.
2% Bovine Serum Albumin (BSA)
To prepare 2% BSA in PBS, dissolve 0.8 g of BSA in 40 mL of 1× PBS.
5% Bovine Serum Albumin (BSA)
To prepare 5% BSA in PBS, dissolve 2.0 g of BSA in 40 mL of 1× PBS.
4′,6-diamidino-2-phenylindole (DAPI) stock solution

Dissolve 5 mg of DAPI in 250 mL of sterile deionized water. Solution should be kept at 4° C. and remains stable for up to 3 weeks. Alternatively, 20 μl aliquots can be stored at −20° C. up to 1 year.

### Laminin 511-Coated Plates

Add 9.6 μL of iMatrix-511 to 1.99 mL of PBS. To coat one well of a 12-well plate add 1 mL of the diluted iMatrix-511 solution. Incubate the plate for 1 h at 37° C., 3 h at room temperature, or overnight at 4° C. Use the plate within 24 h and do not let the plate dry. Parafilm may be used to seal the plate, but is not typically necessary. Aspirate the laminin-511 coating solution before adding cells.

iPSC Passage Medium

iPSC passage media consists of mTeSR medium supplemented with 10 μM Y27632. For example, add 3 μl of 10 mM Y27632 to 3 mL of mTeSR medium. iPSC passage medium should be prepared fresh before each use.

### Podocyte Culture Base Medium

The base media consists of DMEM/F12 with GlutaMax containing 1× B27 serum-free supplement and 1% (v/v) of penicillin-streptomycin. DMEM/F12 with GlutaMax can be stored at 2° C. to 8° C. for up to 12 months. The B27 serum-free supplement can be stored at −20° C. for up to 12 months. Penicillin-streptomycin can be stored at −20° C. for up to 12 months.

### Primitive Streak Induction Medium

The mesoderm induction medium can be prepared by adding 100 ng/ml Activin A, 3 μM CHIR99021, and 10 UM Y27632 to the podocyte culture base medium. Once prepared, primitive streak medium can be stored at 4° C. for up to 1 week.

TABLE 2
Primitive Streak Induction Medium

| | | |
|---|---|---|
| Reagent | Final Concentration | Amount |

| | | | |
|---|---|---|---|
| DMEM/F12 with | n/a | 4.885 | mL |
| GlutaMAX supplement | | | |
| B27 | 1X | 50 | μl |
| Penicillin-Streptomycin | 1X | 50 | μl |

| | | | | |
|---|---|---|---|---|
| Y 27632 | 10 | μM | 5 | μl |
| Activin A | 100 | ng/mL | 5 | μl |
| CHIR99021 | 3 | μM | 5 | μl |

| | | | |
|---|---|---|---|
| Total | n/a | 5 | mL |

### Intermediate Mesoderm Induction Media

The mesoderm induction media can be prepared by adding 8 μM CHIR99021 to the base media. Once prepared intermediate mesoderm induction medium can be stored at 4° C. for a week.

TABLE 3
Intermediate Mesoderm Induction Medium

| Reagent | Final Concentration | Amount | |
|---|---|---|---|
| DMEM/F12 with | n/a | 4.895 | mL |
| GlutaMAX supplement | | | |
| B27 | 1X | 50 | μl |
| Penicillin-Streptomycin | 1X | 50 | μl |
| CHIR99021 | 8 μM | 5 | μl |
| Total | n/a | 5 | mL |

### Nephron Progenitor Induction Medium

The intermediate mesoderm induction medium can be prepared by adding 200 ng/ml FGF9 and 1 μg/mL Heparin to the base media. Once prepared Nephron progenitor induction medium can be stored at 4° C. for a week.

TABLE 4
Nephron Progenitor Induction Medium

| | | |
|---|---|---|
| Reagent | Final Concentration | Amount |

| DMEM/F12 with GlutaMAX | n/a | 4.890 | mL |
|---|---|---|---|
| supplement | | | |
| B27 | 1X | 50 | μl |
| Penicillin-Streptomycin | 1X | 50 | μl |

| | | | | |
|---|---|---|---|---|
| FGF9 | 200 | ng/ml | 5 | μl |
| Heparin | 0.180 | USP/mL | 5 | μl |

| | | | |
|---|---|---|---|
| Total | n/a | 5 | mL |

### Podocyte Induction Medium

The podocyte media can be prepared by adding 100 ng/ml BMP7, 100 ng/ml Activin A, 50 ng/mL VEGF, 3 μM CHIR99021, and 0.1 μM all-trans retinoic acid to the base media. Once prepared, podocyte induction media can be stored at 4° C. for one week.

TABLE 5
Podocyte Induction Medium

| | | |
|---|---|---|
| Reagent | Final Concentration | Amount |

| | | | |
|---|---|---|---|
| DMEM/F12 with | n/a | 4.875 | mL |
| GlutaMAX supplement | | | |
| B27 | 1X | 50 | μl |
| Penicillin-Streptomycin | 1X | 50 | μl |

| | | | | |
|---|---|---|---|---|
| CHIR99021 | 3 | μM | 5 | μl |
| Activin A | 100 | ng/mL | 5 | μl |
| BMP-7 | 100 | ng/mL | 5 | μl |
| VEGF | 50 | ng/mL | 5 | μl |
| all-trans retinoic acid | 0.1 | μM | 5 | μl |

| | | | |
|---|---|---|---|
| Total | n/a | 5 | mL |

Use the suggested amount of media per well of the plate following the manufacturer's instructions. Add e.g. 2 mL of media per well of a 12-well plate.

### Procedure

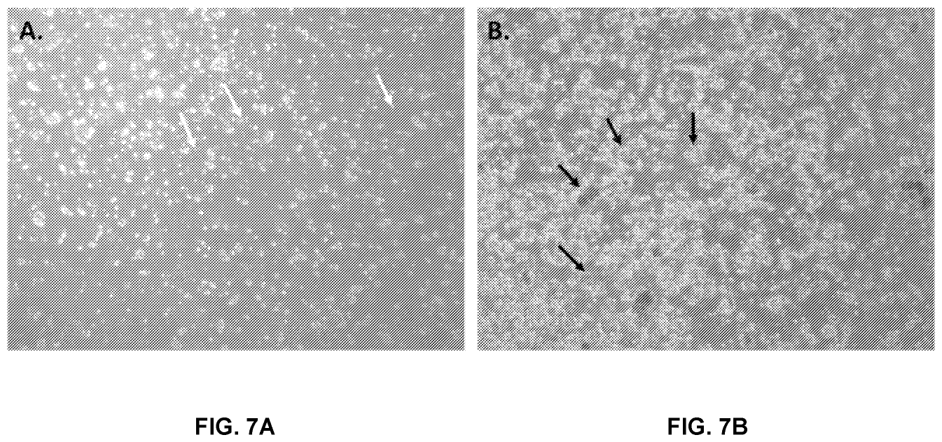

Growing human iPSC in feeder-free culture with mTeSR medium-Timing ˜7d
1. Prepare Geltrex or/Matrigel coated plates (see Reagent Setup)
2. Transfer 5 mL of warmed mTeSR media (See Reagent Setup) into a 15 mL tube.
3. Thaw a frozen vial of hiPSC containing at least 1.5-2.0×10⁶ cells in a 37° C. water bath with gentle shaking until the ice starts to melt.
4. Transfer hiPSCs in a drop-wise manner into the 15 ml conical tube containing prewarmed media (Step 1). Gently rotate the tube to mix the cells with the media. Centrifuge the cells for 5 min at 300× g at room temperature.
5. Remove the supernatant and add 3 mL of fresh iPSC passage media (See Reagent Setup).
6. Seed the cells onto plates coated with Geltrex or/Matrigel i.e., 1.5×10⁶ into one well of a 6 well plate. Make sure that cells are distributed evenly by rocking back and forth gently to distribute the cells and incubate at 37° C. in a 5% CO₂ incubator.
7. After 24 h, change the iPSC passage media to regular mTeSR media.
8. Change the media every day. Changing media every day is preferably since iPSC proliferate quickly. Lack of nutrients in the exhausted media will lead to cell detachment and death. Note: Different iPS cell lines are grown in different starting media such as Stem Flex or, Essential 8. StemFlex and Essential 8 have been used in substitution for mTESR for other cell lines requiring these media types. Cells should be approximately 80-100% confluent after 7 days of culture. If cells do not reach this confluency, allow them to grow until they reach confluency. The cells can be cryopreserved or differentiated when they reach confluency. Follow the instruction below for cryopreservation or differentiation.
   Human iPSC Passage TIMING 30 Min
9. Prepare Geltrex or Matrigel coated plates (See Reagent Setup).
10. Remove the media from the hiPSCs in the 6-well plate.
11. Wash the cells with 3 mL of PBS twice. Aspirate PBS.
12. Add 1 mL per well of Accutase to cells and incubate at 37° C. for 3 min.
13. Add 2 mL per well of warm mTeSR to the cells, mix, and ensure cells have lifted off from the plastic surface. Do not pipette cells more than twice as hiPSCs are very sensitive to mechanical perturbation.
14. Collect cells in a 15 mL tube. Count the cell number using a hemocytometer. Automatic cell counters should be validated with a hemocytometer before use. Some commercial machines do not accurately count iPSC due to their unique shape.
15. Calculate required volume to achieve 1.5×10⁶ iPSC per well of the 6 well plate. Aliquot the iPSC to be plated into a 15 mL tube and centrifuge at 300× g for 5 min.
16. Remove the supernatant and gently resuspend the cells in iPSC passage media (see Reagent Setup).
17. Seed the cells onto the Geltrex or Matrigel coated plates. Make sure that cells are distributed evenly by gently rocking back and forth and incubate at 37° C. in a 5% CO₂ incubator for 24 h.
18. The iPSC will attach and grow colonies and will be 20-30% confluent on Day 1. If the cells do not attach and grow after 24 hours, do not attempt to use the cells for differentiation. Lower cell density leads to spontaneous differentiation of iPSCs.
   Human iPSC Cryopreservation TIMING 30 Min
19. Rinse the cells 3 times with PBS and add 0.5 mL of Accutase per 6-well plate.
20. Incubate the cells at 37° C. and 5% CO₂ for 5 min or until the cells start to detach. Incubate for 2-3 min more if the cells do not start to detach. Do not overdigest the cells with Accutase, as this will result in cell death (FIG. 7).
21. Add 3 mL of fresh mTeSR media to the dissociated cells, mix, and ensure cells have lifted off from the plastic surface. Do not pipette cells more than twice as hiPSCs are very sensitive to mechanical perturbation.
22. Collect cells in a 15 mL tube. Count the cell number using a hemocytometer.
23. Centrifuge the cells for 5 min at 300× g at room temperature.
24. Remove the supernatant and gently resuspend 2×10⁶ cells per mL of a cryopreservation medium consisting of 90% FBS and 10% DMSO. Alternatively, commercially available cryopreservation media such as Cryostor can be used instead.
25. Add 1 mL of cell suspension per cryopreservation tube.
26. Place the tubes into a cryovial freezer box and freeze at −80° C. for 24 h.
27. Transfer the tubes to a liquid nitrogen cell storage tank for long-term cryopreservation. To start a live culture from a frozen vial, follow steps 1-8.
   hiPSC plating for differentiation TIMING ˜1 d, day 0
28. Follow steps from 9-14.
29. Calculate required cell volume to achieve 1×105 iPSC per well of the 12 well plate.
30. Aliquot the iPSC to be plated to a 15 ml tube and centrifuge at 300× g for 5 min.
31. Remove the supernatant and gently resuspend the cells in iPSC passage media (see Reagent Setup).
32. Seed the cells on the laminin-511-coated plates. Make sure that cells are distributed evenly by rocking back and forth and incubate at 37° C. in a 5% CO₂ incubator for 24 h.
33. iPS cells will attach and grow colonies and will be 40-50% confluent on Day 1 (FIG. 2). If the cells have not reached the desired confluency, they may grow for one additional day prior to proceeding.
   Differentiation of Human iPSC into the Posterior Primitive Streak TIMING 2 d, Day 1-3
34. Prepare primitive streak induction media (see Reagent Setup)
35. Aspirate the iPSC passage media from cells and add 2 mL primitive streak media per well of a 12 well plate.
36. Culture the cells in primitive streak media for 2 days without changing the media. 37. Primitive streak cells have a triangular shape morphology are 40-50% confluent (FIG. 2).

### Induction of Intermediate Mesoderm TIMING 3 d, Day 3-6

38. Prepare intermediate mesoderm induction media (see Reagent Setup)
39. Aspirate the primitive streak media from the cells and add 2 mL per 12-well plate intermediate mesoderm media.
40. Refresh medium every day and let the induction of mesoderm continue until day 5.
41. The intermediate mesoderm cells will start to proliferate and reach 55-60% confluency (FIG. 2). Since the cells start to proliferate, the rate of consumption of growth factors and supplements will increase. Make sure to add sufficient media (i.e., 2 mL for each well of a 12 well plate), since the lack of nutrients will lead to cell death.

### Induction of Nephron Progenitor Cells TIMING 2 d, Day 6-8

42. Prepare nephron progenitor differentiation medium (see Reagent Setup).
43. Aspirate the medium from the intermediate mesoderm cells and incubate with 2 mL per 12-well plate of nephron progenitor medium.
44. Refresh medium daily.
45. Culture cells in nephron progenitor medium for 2 days.
46. The intermediate mesoderm cells will start to form clumps and will have 50-60% confluency (FIG. 2). As the cells start to proliferate, the rate of consumption of growth factors and supplements will increase. Make sure to add sufficient nephron progenitor media, since the lack of nutrients will lead to cell death. Note: FGF9 concentration below 200 ng/ml may result in an inefficient induction of the nephron progenitors.

### Derivation of Mature Kidney Podocytes on Laminin-Coated Plates TIMING 5 d, Day 8-13

47. Prepare podocyte induction medium (see Reagent Setup)
48. Remove the intermediate mesoderm differentiation medium from cells and wash cells one time with PBS.
49. Add Accutase (0.5 mL per well for 12 well plates) to cells and incubate for 5 min at 37° C.
50. Visualize cells under the microscope to ensure the cells are properly dissociated into either individual cells or small clumps. It is important to avoid bigger clumps since it leads to poor podocyte differentiation.
51. Pipette the cells gently 3 times and transfer the cells to a 15 mL tube. Add fresh nephron progenitor media to quench the Accutase to the tube.
52. Centrifuge cells at 300× g for 5 min and aspirate the supernatant. Resuspend the cells with podocyte induction media at a 1:4 dilution ratio to plate 2.5×105 cells per well of a laminin-511-coated 12-well plate.
53. Change the podocyte induction media daily.
54. The derived podocytes are characterized by a large cell body and arborized morphology (FIG. 2). The iPSC-derived podocytes may be used for further studies at this point.
   Endpoint Analysis of iPSC-Derived Podocytes by Immunostaining TIMING 2 Days, Day 13
55. Remove the media from the podocytes and wash the cells with PBS twice.
56. Fix the cells in 4% paraformaldehyde (PFA) in PBS by adding 1 mL per well of a 12 well plate for 15 min. Then wash the cells with PBS twice.
57. For intracellular staining, permeabilize the cells with 0.1% Triton X-100 for 10 min.
58. Block the samples by adding 1 mL per well of 12 well plates in PBS-BT for 30 min.
59. Stain with the desired primary antibodies diluted in 2% BSA for either 4 h at RT or 4° C. for overnight (Table 1).
60. Wash the cells with 500 μl PBS per well of 12-well plate twice by aspirating and adding fresh PBS. Incubate the cells with the corresponding secondary antibody (Table 1).
61. Stain the nuclei with DAPI diluted 1:1000 in PBS for 5 min and visualize using a fluorescent microscope.
62. For F-actin staining, fixed cells may be stained with phalloidin and DAPI instead (FIG. 5).
   Endpoint Analysis of iPSC-Derived Podocytes by Flow Cytometry Analysis TIMING 1-2 d, Day 13
63. Remove the media from the podocytes and wash the cells with PBS twice.
64. Add 1 mL of 0.05% trypsin per well of 12 well plates and incubate at 37° C. for 5 min.
65. Add 2 mL of warm DMEM/F12 to the cells, mix, and ensure cells have lifted off from the plastic surface.
66. Collect cells in a 15 mL tube and centrifuge at 300× g for 5 min.
67. Wash the cells with 1 mL of PBS twice. Aspirate PBS.
68. Fix the cells in 1 mL of 4% paraformaldehyde (PFA) in PBS for 15 min. Then wash the cells with 500 μl of 2% BSA twice by centrifugation at 400×g for 2 min after each wash. Remove the supernatant.
69. For intracellular staining, permeabilize the cells with 100% cold ethanol for 10 min, then centrifuge at 400×g for 2 min. Remove the supernatant and wash the cells with 2% BSA for twice with centrifugation. Remove the supernantant.
70. Block the samples by adding 1 mL of 5% BSA in PBS for 30 min, then centrifuge briefly at 400×g for 2 min. Remove the supernatant and wash the cells with 2% BSA for twice with centrifugation. Remove the supernatant.
71. Depending on the secondary antibodies being used, separate the desired isotope controls. These controls are samples that will only be treated with secondary antibody and no primary antibody.
72. Stain with the desired primary antibodies (200 μl) diluted in 2% BSA for either 4 h at RT or 4° C. overnight (Table 1).
73. Wash with 2% BSA and remove the supernatant as in previous steps. Incubate the cells with 200 μl of the corresponding secondary antibody diluted in 2% BSA for 2 h at RT or 4° C. overnight (Table 1).
74. Analyze the cells using a flow cytometer and compare to the negative isotype controls using FlowJo software (FIG. 3C).
   Endpoint Analysis of iPSC-Derived Podocytes by FITC Albumin Uptake Assay TIMING 1 d, Day 13
75. Remove the media from the podocytes and incubate with podocyte induction medium overnight.
76. Wash the cells with PBS two times by aspirating and replacing with fresh PBS.
77. To evaluate the temperature-dependent endocytosis of albumin in podocytes add 50 μg/mL FITC-conjugated bovine serum for either 1 h at 4° C. or 37° C. for 24 h to the podocytes.
78. Visualize endocytosis with a fluorescent microscope.

TABLE 6
List of Antibodies

| | Primary | | | |
|---|---|---|---|---|
| Cell type | Antibody | Origin/Isotype | Supplier/Catalogue # | Dilution |
| Undifferentiated | Oct-4 | Rat IgG2b | R& D | 1:200 |
| | | | systems/MAB1759SP | |
| Primitive streak | MIXL1 | Rabbit IgG | Proteintech/22772-1- | 1:200 |
| | | | AP | |
| Intermediate | PAX8 | Rabbit IgG | Proteintech/10336-1- | 1:200 |
| mesoderm | | | AP | |
| | GATA3 | Goat IgG | R& D | 1:200 |
| | | | systems/AF2605 | |
| Nephron | CITED1 | Mouse IgG2a | Fisher Scientific/89- | 1:200 |
| Progenitors | | | 335-107 | |
| | SIX2 | Rabbit IgG | Proteintech/11562-1- | 1:200 |
| | | | AP | |
| Podocyte | MAFB | Rabbit IgG | Abcam/ab223744 | 1:200 |
| | PODOX | Rabbit IgG | Proteintech/18150-1- | 1:200 |
| | | | AP | |
| | Nephrin | Sheep IgG | R& D | 1:200 |
| | | | systems/AF4269 | |
| | Synaptopodin | Rabbit IgG | Abcam/ab224491 | 1:200 |
| Pathway | F-actin | Phalloidin | molecular grade | 1:100 |
| | | 594 | Probes/A12381 | |

| Secondary | Alexa 488, goat anti-mouse | Life Technologies/A- | 1:200 |
|---|---|---|---|
| | IgGa | 21131 | |
| | Alexa 488, goat anti-rat IgG | Life Technologies/A- | 1:400 |
| | | 11006 | |
| | Alexa 488, donkey anti-goat | Life Technologies/A- | 1:400 |
| | IgG | 11055 | |
| | Alexa 594, goat anti-rabbit | Life Technologies/A- | 1:400 |
| | IgG | 11037 | |
| | Alexa 594, donkey anti-sheep | Life Technologies/A- | 1:400 |
| | IgG | 11016 | |

### Expected Outcomes

This protocol allows the serum-free production of functional podocytes from human iPSC in a fast and efficient manner. The differentiation process begins with the generation of primitive streak cells from iPSC via two days of exposure to activin A and a low dose of Wnt signaling activator CHIR (3 μM). The cells at this stage have a spiky, triangular morphology (FIG. 2). Subsequent treatment of these cells with high-dose CHIR (8 UM) for 3 days produces intermediate mesoderm (FIG. 3). Treatment of the intermediate mesoderm cells with FGF9 for two days permits the generation of nephron progenitors (FIG. 3). Finally, podocytes are derived from nephron progenitor cells through incubation in podocyte induction media containing VEGF for the survival of podocytes, retinoic acid to support the generation of glomerular transition cells, and BMP7 for differentiation and survival.

Higher magnification brightfield images of the derived podocytes show the main body surrounded by structures resembling elongated foot processes (FIG. 4A). Additionally, flow cytometry analysis of the podocytes revealed high expression of markers MAFB (77%) and PODX (58%) indicating efficient differentiation (n=3) (FIG. 4B). To further confirm the reproducibility of the protocol, a MAFB: mTagBFP2 blue fluorescent protein reporter iPSC cell line was used (Vanslambrouck et al., 2019). The differentiated podocytes from this line exhibited the same arborized morphology (FIG. 4C). Flow cytometry analysis found that 37% the cells from this line were BFP+ and 57% cells were MAFB+ (FIG. 4D). The difference in the expression of MAFB suggests that this cell line may require further optimization for efficient differentiation.

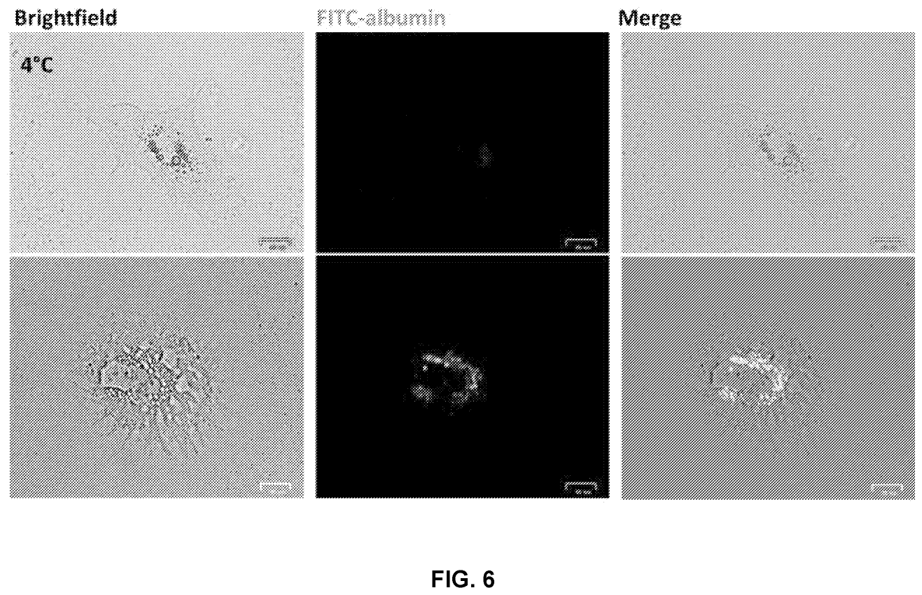

To confirm the phenotype electron microscopy was performed on the derived podocytes. SEM analysis confirmed the arborized structure with thin processes extending to the adjacent cells (FIG. 5B). TEM images showed tight junctions present between the adjacent cells (FIG. 5C). Immunostaining with podocyte markers was performed to evaluate the efficient differentiation of podocytes from iPSCs. The podocytes derived from iPSC stained positive for mature podocyte markers (FIG. 5A). The functionality of the hiPSC-derived podocytes was evaluated by FITC-albumin uptake assay. The results showed that the iPSC-derived podocytes endocytose albumin in a temperature-dependent manner. iPSC-derived podocytes endocytosed albumin in intracellular vesicles at 37° C., whereas the cells at 4° C. displayed inhibited endocytosis (FIG. 6). Together, this protocol provides a feeder-free robust method for the derivation of podocytes from iPSCs in thirteen days with a additional 1 week period after differentiation for experiments. The cells start to lose their proliferating efficiency and lose the structure after a week.

### Applications and Limitations of the Protocol

The protocol recapitulates the natural process of podocyte development, resulting in a culture of cells expressing podocyte-specific markers beginning from human iPSC. The phenotype of the iPSC-derived podocytes is similar to those of terminally differentiated podocytes. Therefore, this protocol could be developed further in order to model podocytopathies, for toxicity screening, or to study podocyte biology. Diseases affecting podocytes include autoimmune disorders (Koffler, D., et al. J Exp Med. 1967 126:607-624; Radford, M. G., et al. J Am Soc Nephrol. 1997 8:199-207; Wilson, C. B., et al. Ann Intern Med. 1972 76:91-94), bacterial endocarditis (Neugarten, J., et al. Am J Kidney Dis. 1984 77:297-304), HIV (Barisoni, L., et al. J Am Soc Nephrol. 1999 10:51-61), Alport syndrome (Kashtan, C. E. J Am Soc Nephrol. 1998 9:1736-1750), and diabetic nephropathy (Sassy-Prigent, C., et al. Diabetes 2000 49:466-475). Starting iPSC of different genetic backgrounds could be derived from fibroblasts or other cells isolated from patients. Alternatively, CRISPR-mediated genetic mutation to generate mutant iPSC lines can model genetic kidney diseases (Freedman, B. S., et al. Nat Commun. 2015 6:8715; Kim, Y. K., et al. Stem Cells. 2017 35 (12): 2366-2378).

Compared to existing methods, this protocol provides a comparable method for podocyte differentiation. Although some protocols report high efficiency, they require prolonged culture time. Nevertheless, the podocytes derived have not matured to the level of the adult kidney, suggesting that further adjustments could improve maturation.

### Troubleshooting

Cells do not Grow Well after Thawing. Step 7

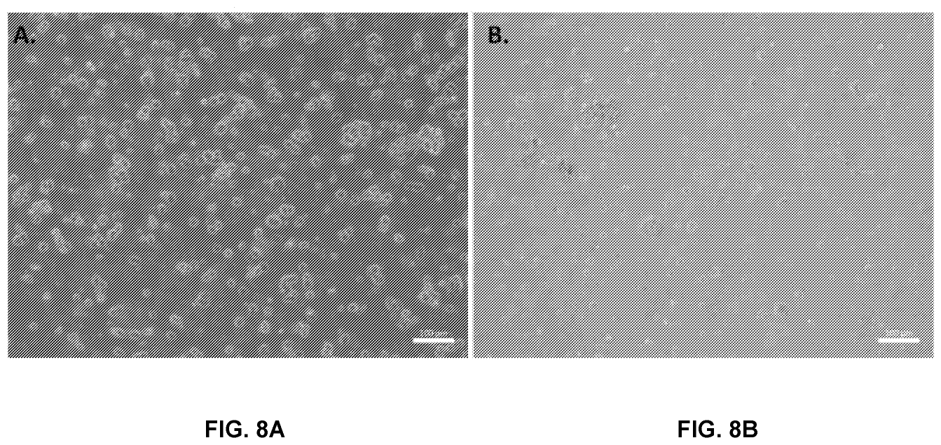

Unhealthy cells after thawing can result from unhealthy cells at cryopreservation (FIG. 8). Only cryopreserve healthy cells or use a different cryopreservation reagent (CryoStor). Because this is a relatively common problem, it is recommended to freeze early passage cells on multiple days for backup purposes.

Cells Detach During the Intermediate Mesoderm Stage. Step 45

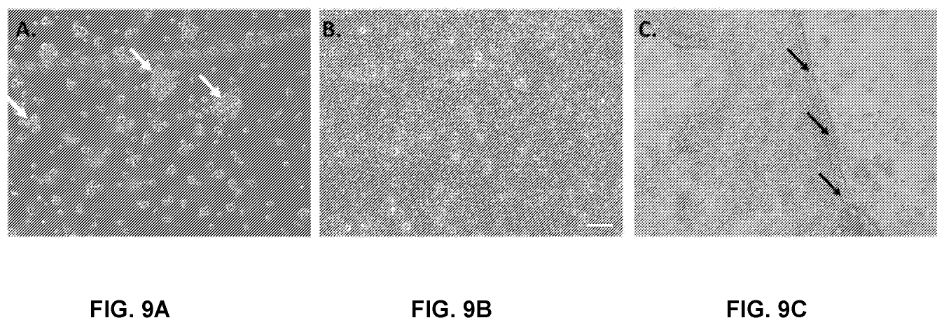

The reason for the cell detachment is either very high or very low cell density (FIG. 9). Please follow the cell number mentioned in Step 13 for plating.

### No Intermediate Mesoderm Marker Expression at Day 6. Step 46

Depending on the cell line used, the intermediate mesoderm induction will need to be adjusted. Optimize the number of days (2-4) of treatment with 8 μM CHIR for the cell line used.

Nephron Progenitors do not Attach and Differentiate into Podocytes at Day 9. Step 53

Pippeting too vigorously while dissociating the progenitors will result in cell damage. Pipette gently while dissociating. Adding 10 UM Y27632 to the podocyte media for the first 24 h may help if gentle pipetting does not solve the problem.

Failure to Visualize Podocyte Markers. Step 61

The reason for this is failed differentiation of the podocytes. Thaw another iPSC vial for the next protocol attempt and confirm the presence of nephron progenitor markers at day 7 by immunostaining. Negative staining for nephron progenitors at day 7 means that the iPS cells may have become aberrantly differentiated, preventing their directed differentiation into the desired podocyte cell type.

### Example 2

FIGS. 9A to 9C show undesirable plating densities of human iPS cells at day 0. FIG. 9A shows iPSC plated at too low density will fail to differentiate. Cells plated such low density will detach (arrows) and start to die following the primitive streak media treatment. FIG. 9B shows high density plating of iPSCs at day 0 will result in detachment of the cells (arrows) after the primitive streak media treatment (FIG. 9C).

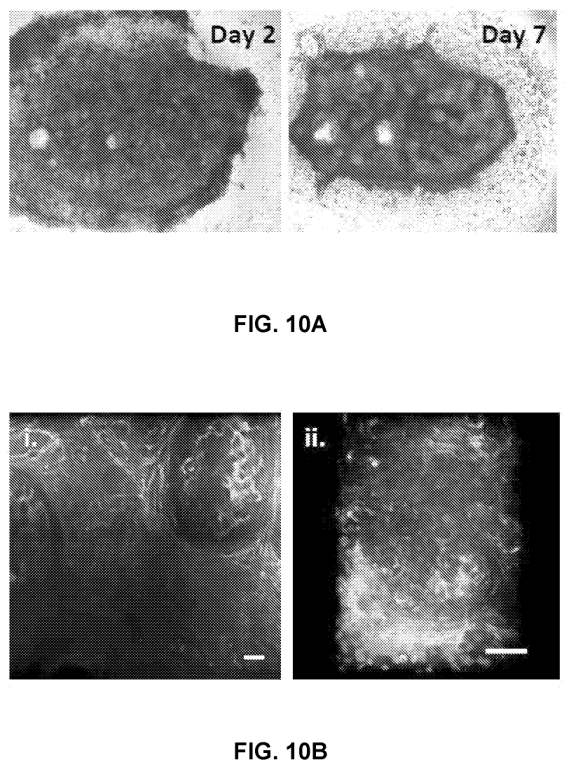

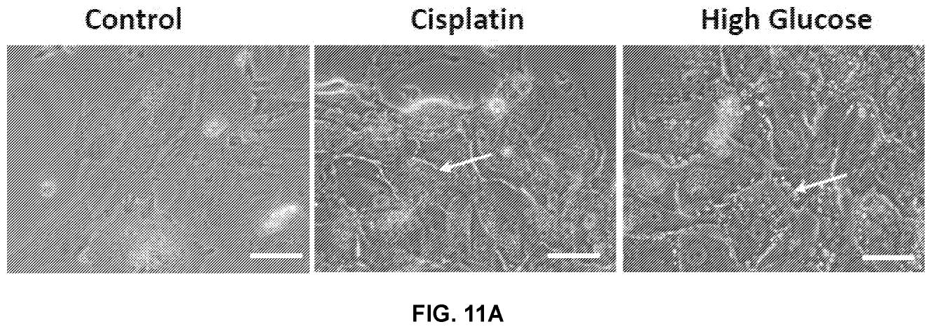

FIGS. 10A and 10B show integration of iPSC-derived podocytes into mouse embryonic kidneys. FIG. 10A contains brightfield images of the recombinant organoids generated using iPSC-podocytes and E12.5 embryonic kidney at day 2 and day 7 after initiation of ex vivo co-culture. FIG. 10B contains higher (40×, i) and lower (20×, ii) magnification images showing the iPSC-derived podocytes (human-specific MAFB+) incorporated into mouse glomerular structures (mouse-specific NPHS1+) (n=2). Counterstaining with DAPI. Scale bar is 50 μm.

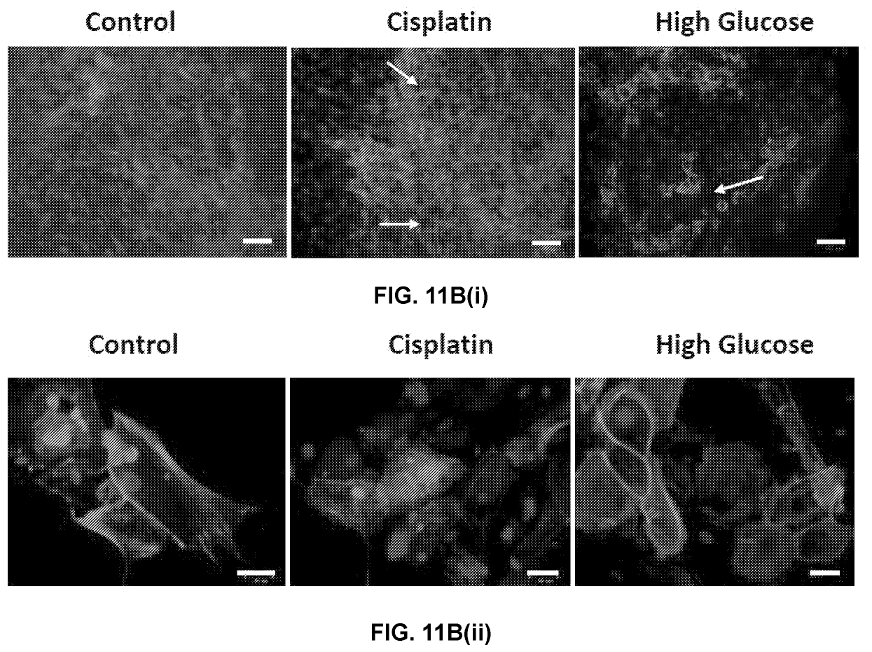

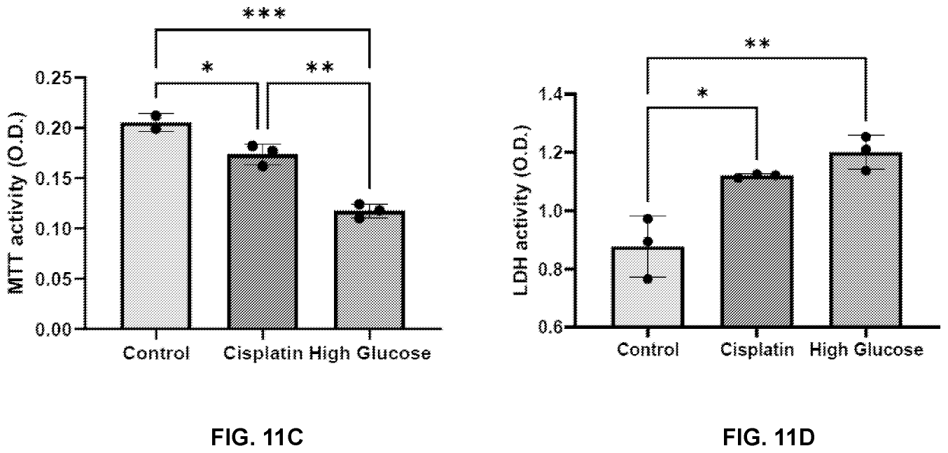

FIGS. 11A to 11D show modeling diabetic kidney disease by high glucose treatment of iPSC-derived podocytes. FIG. 11A contains brightfield images of cisplatin- or high glucose-treated podocytes showing damage to the junctions between cells (arrows). Scale bar represents 50 μm. FIG. 11B (i) shows reorganization of F-actin (phalloidin-red) along the cell periphery after the treatment with either cisplatin (5 μM) or high glucose (100 mM). Scale bar is 100 μm. FIG. 11B (ii) contains higher magnification images showing out-of-order and intertwined actin fibers in treated groups compared to control groups podocytes with paralleled fasciculate models (n=2). Scale bar is 30 μm. FIG. 11C shows viability analysis by MTT assay showing reduction in podocyte viability after treatment with glucose. FIG. 11D shows cytotoxicity analysis using LDH assay indicating increased toxicity within the podocytes treated with glucose. * Indicates p<0.05.

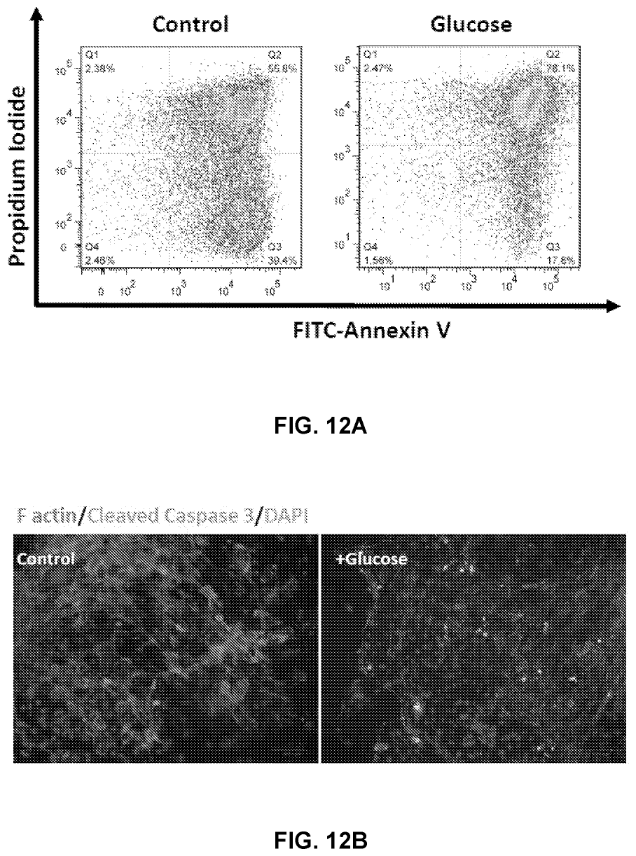

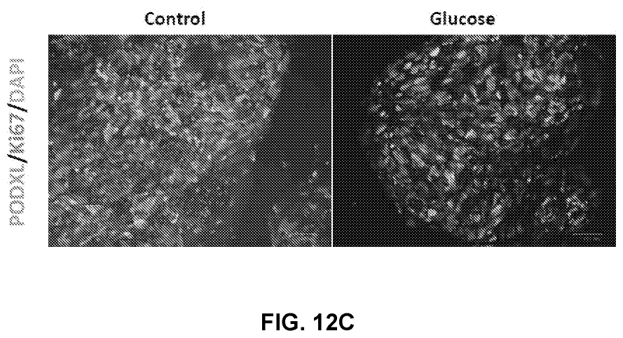

FIGS. 12A to 12 C show modeling diabetic kidney disease by high glucose treatment of iPSC-derived podocytes. FIG. 12A shows Annexin V flow cytometry analayis showing 78.1% of aptoptotic cells in the glucose treated group compared to the 55.8% control untreated group. FIG. 12B are immunostaining images showing increased expression of cleaved caspase 3 in the glucose treated podocytes compared to control group. Scale bar is 100 μm. FIG. 12C are immunostaining images showing decreased expression of proliferation marker Ki67 in the glucose treated podocytes compared to control group. Scale bar is 100 μm.

### Example 3. Podocytes Derived from Human Induced Pluripotent Stem Cells: Characterization, Comparison, and Modeling of Diabetic Kidney Disease

### Background

The discovery of adult human cells that can be reprogrammed into induced pluripotent stem cells (iPSCs) has opened up a new way to generate different cell types in vitro (Takahashi K, et al. Cell 2007 131 (5): 861-72). iPSCs have an unlimited capacity for self-renewal and the ability to develop into most cell types. Many techniques for producing renal cells from iPSCs have been devised based on knowledge gathered from embryonic kidney development (Low J H, et al. Cell Stem Cell. 2019 25 (3): 373-87.e9; Morizane R, et al. Nature protocols. 2017 12 (1): 195-207; Morizane R, et al. Nature Biotechnology. 2015 33 (11): 1193-200). Kidneys are formed from the mesoderm, which develops into the metanephros, from which the ureteric bud and metanephric mesenchyme emerge (McCrory W W. Birth defects original article series. 1974 10 (4): 3-11). The collecting duct, renal pelvis, and ureters develop from the ureteric bud, whereas the renal tubules and glomeruli develop from the metanephric mesenchyme (Xu J, et al. Developmental cell. 2014 31 (4): 434-47). To generate renal cells from iPSCs, most methods rely upon exploiting natural signaling pathways that follow these developmental phases. iPSCs have also been used to create “kidney-in-a-dish” organoids that mimic the growing embryonic kidney (Morizane R, et al. Nature Biotechnology. 2015 33 (11): 1193-200; Higgins J W, et al. bioRxiv. 2018:505396; Kumar S V, et al. Development. 2019 146 (5): dev172361; Takasato M, et al. Nature. 2015 526 (7574): 564-8). However, kidney organoids resemble a human fetal kidney rather than an adult kidney, both morphologically and transcriptionally (Takasato M, et al. Nature. 2015 526 (7574): 564-8). Human kidney organoids contain podocytes, but the high level of cellular heterogeneity makes it difficult to determine the target cell type of an insult (Wu H, et al. Cell Stem Cell. 2018; 23 (6): 869-81.e8; Kim Y K, et al. Stem cells 2017 35 (12): 2366-78). Derived kidney organoids contain 10-20% off-target cells such as neurons. Kidney organoid protocols produce a highly variable number of podocytes, with estimates ranging from 4% to 28% (Wu H, et al. Cell Stem Cell. 2018 23 (6): 869-81.e8).

Several methods for producing monocultures of podocytes from iPSCs have been published throughout the last decade (Musah S, et al. Nat Biomed Eng. 2017 1 (5): 1-12; Bejoy J, et al. STAR protocols. 2021 2 (4): 100898; Qian T, et al. Science Advances. 2017 3 (11): e1701679; Song B, et al. PloS one. 2012 7 (9): e46453; Ciampi O, et al. Stem Cell Res. 2016 17 (1): 130-9). Other available methods require a lengthy culture time or expensive medium components, but a detailed method was recently published that required less culture time and lower-cost components (Bejoy J, et al. STAR protocols. 2021 2 (4): 100898). Despite a proliferation of protocols in recent years, the comparative quality and marker expression of iPSC-derived podocytes obtained from each method was unknown. In this study, four methods were compared for the differentiation of podocytes from iPSCs, including the disclosed method (Bejoy J, et al. STAR protocols. 2021 2 (4): 100898). In addition, the applications of iPSC-derived podocytes were explore in in vitro systems to study podocyte disease, specifically diabetic kidney disease.

Glomerular diseases are linked to changes in the phenotype of proliferating podocytes. Podocytes are highly differentiated cells that attach to capillaries, forming an important part of the nephron's glomerular filtration barrier (GFB) (Reiser J, et al. F1000Res. 2016 5: F1000 Faculty Rev-114). These specialized pericytes have foot-like extensions called foot processes joined by slit diaphragms (Ryan G B, et al. Kidney International. 1976 9 (1): 36-45). Podocyte dysfunction contributes to proteinuria through dedifferentiation, podocyte apoptosis, proliferation arrest, and foot process effacement (Barisoni L, et al. J Am Soc Nephrol. 1999 10 (1): 51-61). As glomerular diseases progress, loss of the podocyte foot process is accompanied by loss of podocyte markers such as Podocalyxin (PODXL) and Synaptopodin (SYNPO). Podocyte loss may be caused by a genetic mutation (Chugh S S. Transl Res. 2007 149 (5): 237-42), diabetic nephropathy (Li J, et al. Kidney International. 2007 72: S36-S42), or nephrotoxic compounds (Hirschberg R. Curr Opin Support Palliat Care. 2012 6 (3): 342-7). Because fully differentiated podocytes have limited proliferative capacity, their loss or injury causes GFB leakage leading to proteinuria and end-stage renal disease (Nangaku M. J Am Soc Nephrol. 2006 17 (1): 17-25; Boute N, et al. Nature genetics. 2000 24 (4): 349-54; Foley R N, et al. Am J Kidney Dis. 1998 32 (5): S112-S9).

During kidney development, the crescent-shaped epithelial cells beneath the growing glomerulus differentiate into podocytes (Pavenstädt H, et al. Physiol Rev. 2003 83 (1): 253-307). Podocyte precursors are polygonal cells that divide quickly and are joined by apical connections. At this stage, the cells express the tight junction protein Zonula occludens-1 (ZO-1) and the early podocyte marker PODXL (Schnabel E, et al. J Cell Biol. 1990 111 (3): 1255-63). ZO-1 migrates from the apical to the basal region as podocytes differentiate, and the foot process and slit membrane develop (28), expressing slit membrane-associated proteins such as Nephrin (NPHS1) (Kawachi H, et al. Kidney international. 2000 57 (5): 1949-61), Podocin (NPHS2), and CD2 Associated Protein. Macromolecules are filtered through the slit diaphragm, which is controlled by tight junction proteins (Schwarz K, et al. The Journal of clinical investigation. 2001 108 (11): 1621-9). During differentiation, several podocyte marker proteins, including SYNPO (Mundel P, et al. J Cell Biol. 1997 139 (1): 193-204) and PODXL (Schnabel E, et al. Eur J Cell Biol. 1989 48 (2): 313-26), increase in expression.

As animal models do not perfectly mimic human disease phenotypes (Pippin J W, et al. Am J Physiol Renal Physiol. 2009 296 (2): F213-F29), methods for producing human podocytes in vitro provide an important approach for uncovering the mechanism of various podocytopathies. In vitro culture of podocytes was introduced in the mid-1970s using cells taken from the renal cortex (Fish A, et al. Laboratory investigation a journal of technical methods. 1975 33 (3): 330-41; Striker G, et al. Transplant Proc. 1980). Podocytes were extracted by sieving the cultured outgrowth of the isolated glomerulus (Takeuchi A, et al. Am J Pathol. 1992 141 (1): 107) or by digesting the entire glomerulus (Harper P A, et al. Kidney Int. 1984 26 (6): 875-80) but rapid dedifferentiation of cells in vitro was a key drawback of this approach. The shape of these podocytes changed from arborized to cobblestone, indicating a reversion to an immature podocyte phenotype (Shankland S J, et al. Kidney Int. 2007 72 (1): 26-36). The only option to maintain the adult phenotype was to isolate fresh cells regularly. To increase proliferative capacity, exogenous human telomerase reverse transcriptase or SV40 big T antigen can immortalize primary cells (Bryan™, et al. Crit Rev Oncog. 1994 5 (4): 331-57; Lee K M, et al. Cytotechnology. 2004 45 (1-2): 33-8), but this causes their function to be compromised and their morphology to be immature. Although iPSC-derived podocytes have many advantages for modeling of podocytopathies, their wide adoption has been hindered by the absence of a comparison study to determine the similarities and differences between diverse protocols that all produce cells with podocyte markers. In this study, podocytes derived from iPSC were compared and their niche specification and utility explored for disease modeling.

### Methods

Differentiation of Podocytes from iPSCs

### Ciampi Protocol

An established protocol was used the to derive the podocytes (Ciampi O, et al. Stem Cell Res. 2016 17 (1): 130-9). iPSCs were grown on Matrigel-coated (Fisher Scientific, Hampton, NH) dishes at a density of 30,000-50,000 cells/cm² in mTesR medium (STEMCELL Technologies, Cambridge, MA) with 10 UM Rho-associated kinase inhibitor Y-27632 dihydrochloride (ROCKi) (STEMCELL Technologies) for 24 h. On Day 1, cells were treated with a stage I medium comprised of a 1:1 mixture of Dulbecco's Modified Eagle Medium/Nutrient mixture F12 (DMEM/F12) plus GlutaMax (Thermo Fisher Scientific, Waltham, MA) and neurobasal media (Thermo Fisher Scientific) with Neuro-2 (N2; Thermo Fisher Scientific) and 1×B27 (Thermo Fisher Scientific), supplemented with 1 μM CHIR99021 (Reagents Direct, Encinitas, CA) instead of 1 μM CP21R7 and 25 ng/ml bone morphogenic protein 4 (BMP4; R&D Systems, Minneapolis, MN). At Day 4, the cells were treated with stage II STEMdiff Albumin Polyvinyl Alcohol Essential Lipids (APEL) medium (STEMCELL Technologies) in the presence of growth factors including 100 nM retinoic acid (RA; STEMCELL Technologies), 50 ng/ml BMP7 (R&D Systems), and 200 ng/ml fibroblast growth factor 9 (FGF9; Peprotech, Cranbury, NJ) for 2 days. On Day 6, cells were dissociated using Accutase (Thermo Fisher Scientific) and 20,000/40,000 cells/cm² were plated on type I collagen-coated plates (Thermo Fisher Scientific). The cells were treated for 7 more days with stage III VRAD podocyte-maintaining medium (DMEM/F12 plus GlutaMax, 10% fetal bovine serum (FBS) (Life Technologies, Carlsbad, CA), 80 UM RA (STEMCELL Technologies), and 100 nM Vitamin D3 (Thermo Fisher Scientific).

### Rauch Protocol

An established protocol was used with optimization to derive the podocytes (Rauch C, et al. PloS one. 2018 13 (9): e0203869-e). iPSCs were seeded onto Geltrex-coated (Fisher Scientific) plates at a density of 9000 cells/cm² and cultured in differentiation medium (medium M1) for 24 h. On day 0, cells received medium consisting of DMEM/Ham F-12 (Thermo Fisher Scientific) with 1.25% FBS, 100 UM non-essential amino acids (NEAA) (Thermo Fisher Scientific), and penicillin/streptomycin (P/S) (Thermo Fisher Scientific) with 5 μM ROCKi. Then from day 1 to day 10 the cells were cultured in medium M1 consisting of DMEM/Ham F-12, 1.25% FBS, 100 μM NEAA with 10 ng/ml Activin A (Peprotech), 15 ng/ml BMP7, and 100 nM RA. For the following ten days (day 11-day 20), differentiated podocyte-like cells were maintained using basic differentiation media devoid of differentiation factors (DMEM/F12, 2.5% FBS, 100 μM NEAA, 1×P/S).

### Musah Protocol

The podocytes were generated using the established protocol with slight modification (Musah S, et al. Nat Biomed Eng. 2017 1 (5): 1-12). Briefly, iPSCs grown until 80% confluency were dissociated using Accutase. The cells were then seeded onto laminin-511 (Peprotech) coated plates and cultured in mTesR media with 10 UM ROCKi for the first 24 h. Then from Day 1 to Day 3 the cells were treated with Stage I medium, also called mesoderm differentiation medium, consisting of DMEM/F12 with GlutaMax supplemented with 100 ng/ml Activin A, 3 μM CHIR99021 and 1× concentration of B27 serum-free supplement. From Day 3 to Day 16, the cells were grown in Stage II medium, called intermediate mesoderm induction medium, consisting of DMEM/F12 with GlutaMax supplemented with 100 ng/ml BMP7, 3 μM CHIR99021, and 1× concentration of B27 serum-free supplement. At Day 16, the intermediate mesoderm cells were dissociated and replated into a laminin-511-coated plates and podocyte phenotype was induced using Stage III medium. Stage III medium consists of DMEM/F12 with GlutaMax supplemented with 100 ng/ml BMP7, 100 ng/ml Activin A, 50 ng/ml VEGF (Peprotech), 3 μM CHIR99021, 0.1 μM RA, and 1×B27 serum-free supplement.

### Bejoy Protocol

iPSCs seeded onto Geltrex-coated plates at a density of 100,000 cells/cm² were treated with mTesR media with 10 μM ROCKi for the first 24 h. On Day 0, cells were treated with base media (DMEM/F12 with GlutaMax, 1×B27) supplemented with primitive streak induction factors including 100 ng/ml Activin A and 3 μM CHIR99021. On Day 2, cells were treated with base media including 8 μM CHIR99021 to induce intermediate mesoderm until Day 5, followed by treatment with base medium containing 200 ng/ml FGF9 and 1 mg/ml Heparin (Sigma-Aldrich, St. Louis, Missouri). On Day 7, to induce the nephron progenitors, the cells were dissociated using Accutase and replated (1:4) onto laminin-511-coated plates. The podocyte phenotype was induced using base media supplemented with 100 ng/ml BMP7, 100 ng/ml Activin A, 50 ng/ml VEGF, 3 μM CHIR99021, and 0.1 μM RA and cells were assayed on Day 12. The detailed methods for deriving the podocytes was carefully described elsewhere (Bejoy J, et al. STAR protocols. 2021 2 (4): 100898).

### Immunocytochemistry

The cells were fixed using 4% paraformaldehyde (PFA) (Thermo Fisher Scientific) and permeabilized with 0.1% Triton X 100 (Sigma-Aldrich). The samples were then blocked and stained with primary antibodies either for 4 h at room temperature (RT) or overnight at 4° C. (Table 7). The cells were then washed and incubated with the corresponding secondary antibody (Table 7). The samples were then stained with DAPI and visualized on either the DM6000 fluorescent microscope (Leica Microsystems, Wetzlar, Germany) or the ZOE™ Fluorescent Imager (Bio-Rad Laboratories, Hercules, CA). Fixed cells were stained using phalloidin to visualize filamentous-actin (F-actin) and 4′, 6′-Diamidino-2-Phenylindole (DAPI) to stain the nucleus. Fluorescent images were analyzed using ImageJ software.

### Flow Cytometry

To evaluate the expression of proteins of interest quantitatively, the cells were harvested by Accutase treatment and analyzed by flow cytometry. Approximately 1×10⁶ cells per sample were first fixed with 4% PFA and washed with staining buffer (2% FBS in phosphate buffered saline (PBS)). The cells were permeabilized with 100% cold methanol, blocked, and incubated with primary antibodies followed by the corresponding secondary antibody. The cells were acquired with BD FACSCanto II flow cytometer (BD Biosciences, Beckton, New Jersey) and analyzed against isotype controls using FlowJo software.

TABLE 7
Primary antibodies, secondary antibodies, and stains for flow
cytometry, immunostaining, and immunoblotting experiments.
Primary Antibodies

| Cell type | Target | Origin | Isotype | Company | Catalog # | Dilution |
|---|---|---|---|---|---|---|
| Undifferentiated | OCT-4 | Rat | IgG2b | R&D systems | MAB1759SP | 1:200 |
| Primitive streak | MIXL1 | Rabbit | IgG | Proteintech | 22772-1-AP | 1:200 |
| Intermediate | PAX8 | Rabbit | IgG | Proteintech | 10336-1-AP | 1:200 |
| mesoderm | | | | | | |
| Nephron | CITED1 | Mouse | IgG2a | Fisher Scientific | 11562-1-AP | 1:200 |
| Progenitors | SIX2 | Rabbit | IgG | Proteintech | 11562-1-AP | 1:200 |
| Podocytes | PODXL | Rabbit | IgG | Proteintech | 18150-1-AP | 1:200 |
| | NEPHRIN | Sheep | IgG | R&D systems | AF4269 | 1:200 |
| | (NPHS1) | | | | | |
| | SYNPO | Rabbit | IgG | Abcam | ab224491 | 1:200 |
| Podocytes | NEPHRIN | Mouse | IgG | R&D Systems | AF3159-SP | 1:200 |
| (Mouse specific) | (NPHS1) | | | | | |
| Podocytes | MAFB | Rabbit | IgG | Abcam | ab223744 | 1:200 |
| (Human specific) | | | | | | |

Stains

| Visualization of | Binds to | Stain | Channel | Company | Catalog # | Dilution |
|---|---|---|---|---|---|---|
| Cytoskeleton | F-actin | Phalloidin | 594 | Molecular | A12381 | 1:00 |
| | | | | Probes | | |
| Nuclei | DNA | DAPI | blue | Millipore Sigma | D9542 | 1:1000 |

Secondary Antibodies

| Species | Fluorophore | Target | Company | Catalog # | Dilution |
|---|---|---|---|---|---|
| goat | Alexa 488 | anti-mouse IgGa | Life Technologies | A-21131 | 1:200 |
| goat | Alexa 488 | anti-rat IgG | Life Technologies | A-11006 | 1:400 |
| goat | Alexa 594 | anti-rabbit IgG | Life Technologies | A-11037 | 1:400 |
| donkey | Alexa 594 | anti-sheep IgG | Life Technologies | A-11016 | 1:400 |

### Western Blotting

Cells grown on a monolayer were washed with cold 1× PBS and scraped in order to be transferred into microfuge tubes kept on ice. The cells were pelleted using a refrigerated microfuge at 500× g for 5 min and lysed in cold lysis buffer (radioimmunoprecipitation assay (RIPA) buffer (Sigma-Aldrich), 100× protease inhibitor mix (1× PI, Sigma-Aldrich), 20× PhosStop tablets phosphatase inhibitors (Roche, Basel, Switzerland). Samples containing 10 μg total protein were loaded onto 4-12% Bis/Tris gels (Life Technologies) and run at 170V for approximately 1 h before transferring to nitrocellulose with the iBlot (Thermo Fisher Scientific). After transfer, blots were blocked for 1 h at room temperature with rocking in 10 ml Tris-buffered Saline (TBS)-based Odyssey Blocking Buffer (TOBB; LI-COR Biosciences, Lincoln, NE). The blots were then rinsed in milli-Q H₂O for 5 min at room temperature with rocking. Then, 5 ml of the primary antibody dilution made in TOBB+0.2% Tween 20 (Sigma-Aldrich) (TOBBT) was added and the blot was incubated with rocking at 4° C. The blots were then washed 3× with 1× TBS (LI-COR Biosciences)+0.1% Tween 20 (TBST). Then 10 ml/blot secondary antibody diluted in TOBBT+0.01% sodium dodecyl sulfate (KD Medical, Columbia, Maryland) was added to each blot and incubated with rocking for 1 h at RT. Blots were washed again 3× with TBST, then 2× with TBS. The blots were scanned using the Licor Odyssey (LI-COR Biosciences) instrument. The expression was quantified using ImageJ software.

### Albumin Uptake Assay

The functionality of derived podocytes was measured using an albumin uptake assay (Song B, et al. PloS one. 2012 7 (9): e46453; Rauch C, et al. PloS one. 2018 13 (9): e0203869-e). Briefly, differentiated podocyte cultures were cultured in serum-free media for 24 h. The next day, cells were rinsed with PBS and then incubated with 50 μg/ml fluorescein isothiocyanate (FITC)-conjugated bovine serum albumin (Thermo Scientific). For the albumin binding assay, the cells were incubated for 1 h at 4° C. To evaluate binding and endocytosis, cells were kept at 37° C. for 24 h. Brightfield images and FITC images were taken using a ZOE fluorescence microscope (Bio-Rad Laboratories) and merging of the images were used to determine the albumin uptake.

### Transcript Measurements

The total RNA of cells at different stages of differentiation was purified using the RNeasy kit by following the manufacturer's protocol (Qiagen, Germantown, MD). cDNA was synthesized by reverse transcription of 1 μg of each RNA sample with the iScript cDNA synthesis kit (Bio-Rad Laboratories). Primers specific for target genes (Table 8) were purchased commercially (Real Time Primers, LLC, Melrose Park, PA).

The gene Glyceraldehyde-3-Phosphate Dehydrogenase (GAPDH) was used as an endogenous control for normalization of expression levels. Real-time reverse transcription-polymerase chain reaction (RT-PCR) was performed on each sample using SYBR Green PCR Master Mix (Bio-Rad Laboratories). The amplification reactions were performed as follows: 2 min at 50° C., 10 min at 95° C., and 40 cycles of 95° C. for 15 s and 55° C. for 30 s, and 68° C. for 30 s. Fold variation in gene expression was quantified by means of the comparative Ct method.

TABLE 8
Primer sequences to detect podocyte transcripts by RT-PCR.

| Primer | Forward Sequence | Reverse Sequence |
|---|---|---|
| SYNPO | GATGTCAACCAAAACCTTGC | GTGCCATTAGATGGGAGTTG |
| | (SEQ ID NO: 1) | (SEQ ID NO: 2) |
| PODXL | AGGCTTGAGTGAGGTGTTTG | AGCCTTTGATTGATTTGCAG |
| | (SEQ ID NO: 3) | (SEQ ID NO: 4) |
| WT1 | TCATCACTGGGAGTGTCCTT | TGGATTTCCTCACCCAGTAA |
| | (SEQ ID NO: 5) | (SEQ ID NO: 6) |
| GAPDH | CTCTCTGCTCCTCCTGTTCGA | TGAGCGATGTGGCTCGGC |
| | (SEQ ID NO: 7) | (SEQ ID NO: 8) |

### MTT Assay

The viability of cells in each sample was measured by MTT assay. Briefly, a MTT reaction mixture was prepared, and filter sterilized followed by a dilution to 1 mg/ml in HBSS without calcium and magnesium (Fisher Scientific). The monolayer cells were spun at 800×g for 2 min at room temperature. Then the media was removed from the wells and 50 μl of 1 mg/ml MTT reagent (Sigma-Aldrich) was added to each well. The solution was gently mixed and incubated at 37° C. in a CO₂ incubator for 1-2 h. After 2 h, the cells were spun and the MTT supernatant was removed. Next, 100 UL of isopropanol was added to cells and mixed vigorously on an orbital shaker to dissolve the MTT formazan precipitate. The optical density (OD) values were read at 560 and 690 nm. The 690 nm reading was used for background correction.

### Cytotoxicity Assay

Cytotoxicity Assay kit G1780 (Promega, Madison, WI) was used to assay the LDH in the media (Promega). Briefly, the cell culture supernatant was collected and spun at 800×g for 2 min and 50 μL of the supernatant was added to a new 96 well plate. Next, 50 μL/well of CytoTox Assay reagent at RT was added to wells containing media. The plate was incubated in the dark at RT for 30 min. Then, 50 μL 1 M acetic acid stop solution was added to each well and the absorbance was read at 490 nm within 1 h. Average readings from media-only wells were used for blank correction.

### Recombination Assay

Cited1-CreER™-GFP transgenic dams pregnant with embryonic day (E) 12.5-15.5 embryos were sacrificed. Two or three embryonic kidneys were dissected, minced, and transferred into Eppendorf tubes for reaggregation. Accutase was used to dissociate the tissues, which were subsequently centrifuged for 5 min to isolate the pellets. The pellet was then mixed well with 50,000 podocytes before being centrifuged again to produce aggregates. The pellet was then transferred to a transwell plate with a Polyether sulfone membrane (0.4 μm) and cultured as organoids. The cells were treated with DMEM/F12 medium with 10% Fetal calf serum (FCS) (Thermofisher Scientific) and incubated for 7 days at 5% CO₂, 37° C. The kidney explants were fixed with 4% PFA for immunostaining on day 7. The fixed samples were stained following the steps of immunocytochemistry mentioned above.

### Statistics

When two groups were compared, the Student's t-test was used. If more than two groups were analyzed, the ANOVA with the Bonferroni post-test was used. p-values that are less than 0.05 were considered to be statistically significant.

### Results

Differentiation of iPSCs to primitive streak, intermediate mesoderm, nephron progenitors, and podocytes

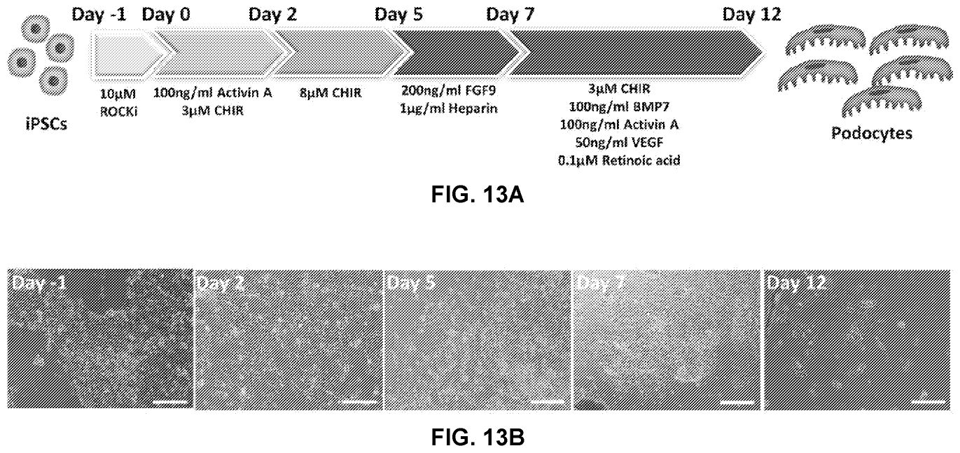

The accelerated method follows a four-stage process of directed differentiation for converting iPSCs into podocytes (Bejoy J, et al. STAR protocols. 2021 2 (4): 100898). Podocytes were derived from iPSCs via stepwise generation of primitive streak-like cells, intermediate mesoderm cells, proliferative nephron progenitors, and finally podocytes (FIG. 13A).

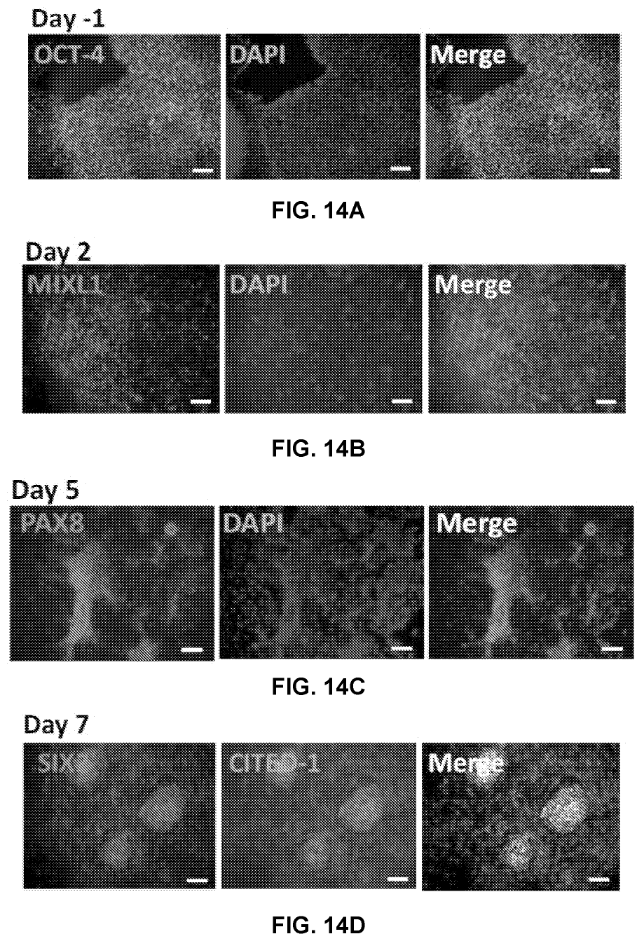

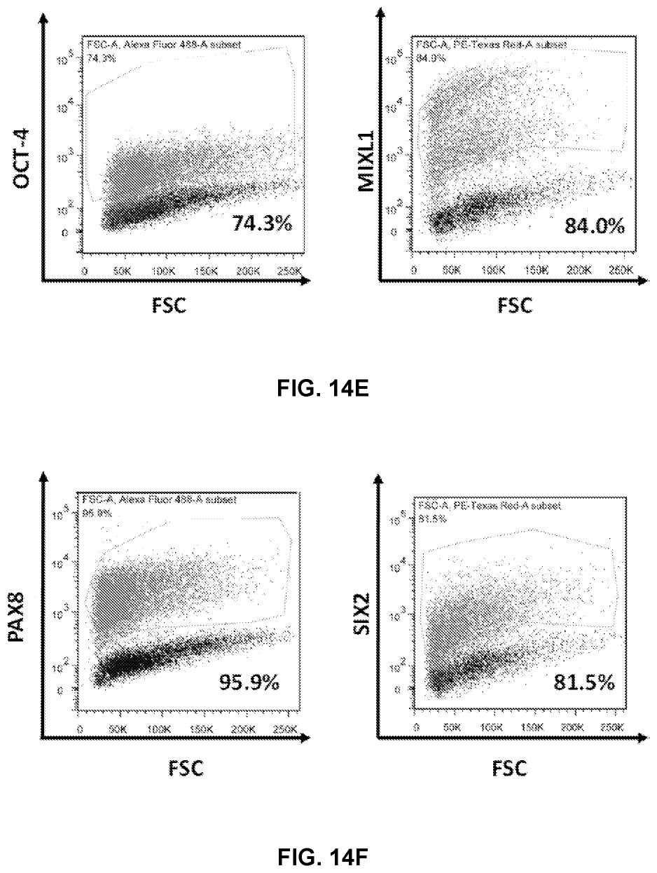

The differentiation protocol began by confirming iPSC pluripotency. The iPSCs possessed a flat, small, and round morphology with well-defined edges (FIG. 13B). iPSCs were also stained for OCT4 (POU5F1) to verify pluripotency (FIG. 14A). Wingless-related integration site (Wnt) signaling combined with activin signaling has been shown to generate the primitive streak (Qian T, et al. Science Advances. 2017 3 (11): e1701679; Lian X, et al. Nature methods. 2015 12 (7): 595-6). Therefore, iPSCs were treated for two days with Activin A and the small molecule Wnt signaling activator CHIR99021 (CHIR). Flow cytometry analysis revealed that octamer-binding transcription factor 4 (OCT4) expression dramatically decreased after initiation of differentiation (FIG. 14E, 19A). The resulting cells uniformly expressed primitive streak marker Mix Paired-Like Homeobox (MIXL1) by immunostaining and flow cytometry analysis (FIG. 14B, 14F). Prolonged activation of Wnt signaling supports iPSC differentiation into the intermediate mesoderm (Takasato M, et al. Nature. 2015 526 (7574): 564-8). Primitive streak was treated with a higher concentration of CHIR99021 (8 μM from 3 M) for 2-3 days to induce the differentiation of PAX8+ intermediate mesoderm cells (FIG. 14C) and reduce the population of MIXL1+ cells to 26.7%. Flow cytometry demonstrated that 95.9% of cells expressed PAX8 at day 5 following the differentiation to the intermediate mesoderm stage (FIG. 14G).

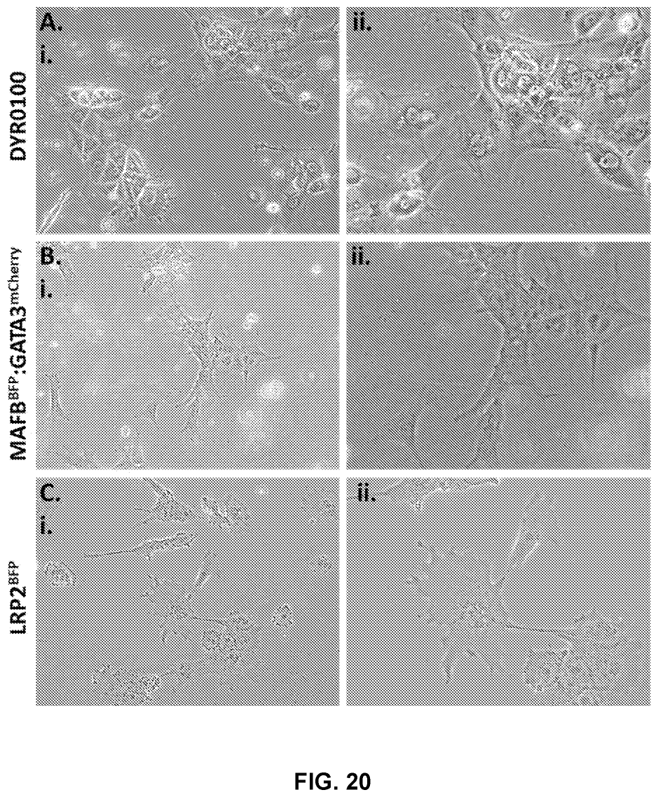

Since a high concentration of FGF9 has been demonstrated to generate nephron progenitors from intermediate mesoderm cells (Takasato M, et al. Nature. 2015 526 (7574): 564-8; Ciampi O, et al. Stem Cell Res. 2016 17 (1): 130-9), the same concentration of FGF9 (200 ng/ml) (Takasato M, et al. Nature. 2015 526 (7574): 564-8) along with heparin (1 μg/ml) was added to stabilize FGF9. At day 7 of differentiation, the nephron progenitors took on a cobblestone-like appearance and expressed nephron progenitor markers SIX Homeobox 2 (SIX2) and Cbp/p300 interacting transactivator with Glu/Asp rich carboxy-terminal domain 1 (CITED1) by immunostaining (FIG. 14D) and flow cytometry analysis (FIG. 14H). Activation of various cell signaling pathways are required for the generation and maintenance of podocytes during kidney development including BMP7 (Ciampi O, et al. Stem Cell Res. 2016 17 (1): 130-9), retinoic acid (Duester G. Cell. 2008 134 (6): 921-31), and vascular endothelial growth factor (VEGF) (Eremina V, et al. Curr Opin Nephrol Hypertens. 2004 13 (1): 9-15). Nephron progenitors were treated with the Musah podocyte media containing a cocktail of growth factors for 5 days (Musah S, et al. Nat Biomed Eng. 2017 1 (5): 1-12). The resulting iPSC-derived podocytes showed large, arborized morphology at day 12 (FIG. 13B). The podocyte differentiation protocol was repeated on three independently-derived iPSC lines from two genetic backgrounds: DYR0100 (origin: SCRC-1041 foreskin fibroblast cell line), MAFB: mTagBFP2/GATA3mCherry (origin: CRL-2429 foreskin fibroblast cell line), and LRP2: mTagBFP2 (origin: CRL-2429 foreskin fibroblast cell line) (Bhargava N, et al. bioRxiv. 2021 2021.07.31.453934; Vanslambrouck J M, et al. J Am Soc Nephrol. 2019 30 (10): 1811-23; López-Muneta L, et al. Front Cell Dev Biol. 2022 9; Castro-Viñuelas R, et al. Mol Ther. 2021 23:569-81) (FIG. 20). The CHIR99021 treatment time may need to be adjusted between 2-3 days depending on the cell line used.

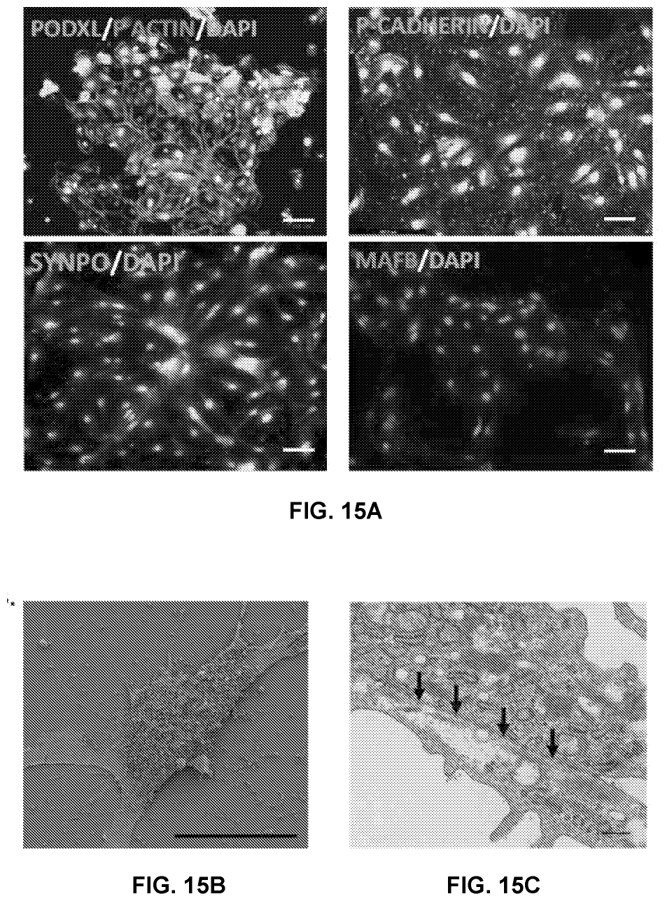

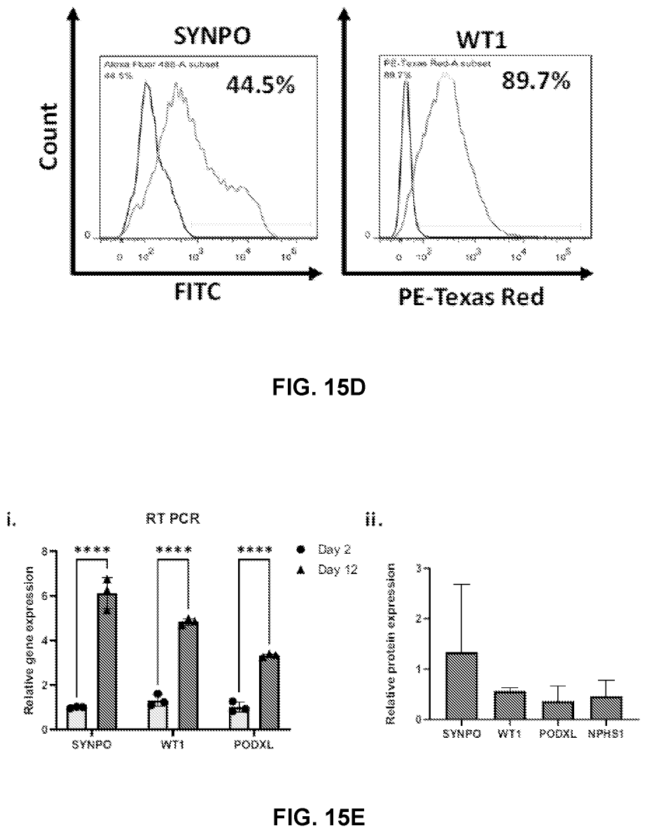

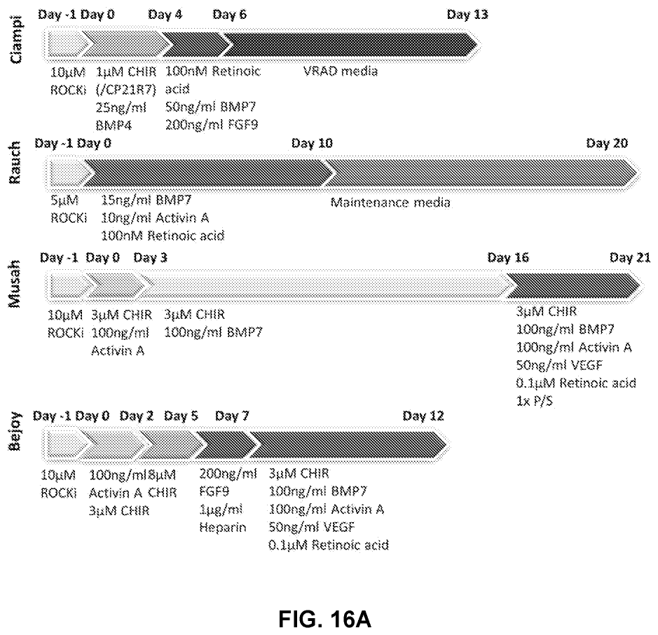

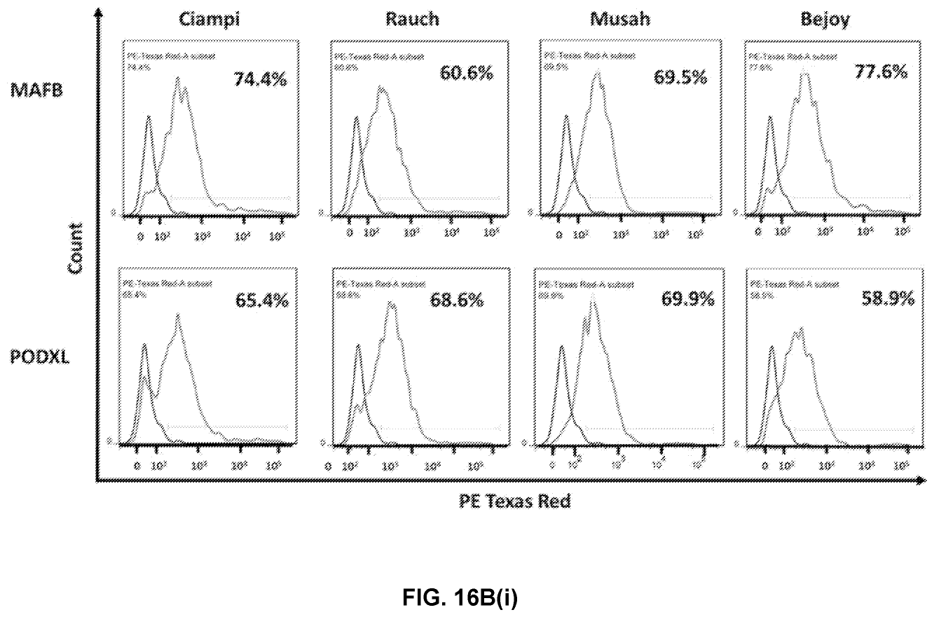

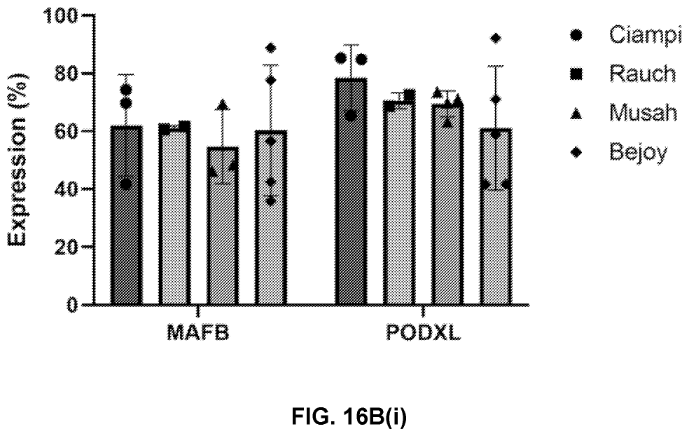

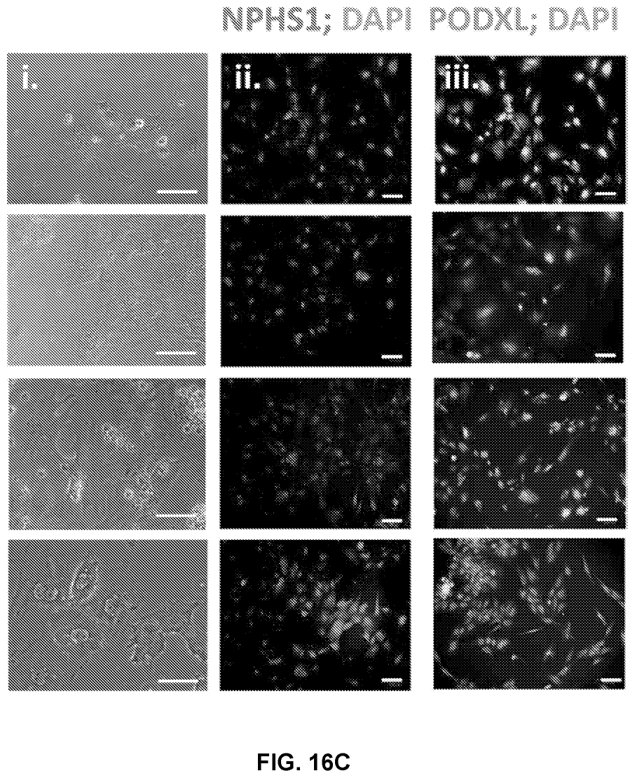

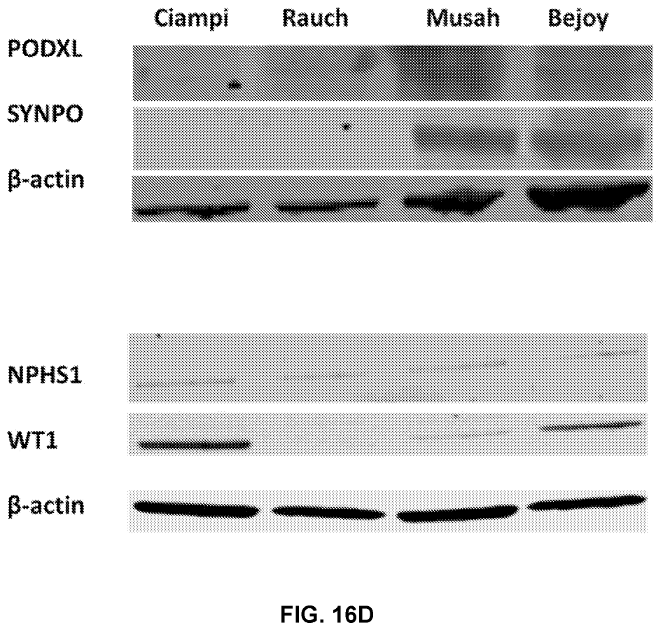

Immunostaining of day 12 podocytes showed expression of podocyte markers musculoaponeurotic fibrosarcoma oncogene family, B (MAFB), PODXL, SYNPO, NPHS1 and placental cadherin (P-cadherin; FIG. 15A and FIG. 16C). Fluorescent-labeled phalloidin was used to visualize F-actin cytoskeletal bundles in differentiated podocytes (FIG. 15A). Flow cytometry analysis demonstrate that derived podocytes expressed Wilms' tumor suppressor gene 1 (WT1) (89.7%), MAFB (77.6%), and PODXL (58.6%), indicating efficient production of podocytes from iPSCs (FIG. 15D and FIG. 16B). The expression of podocyte-associated transcripts throughout the differentiation process was also characterized by RT-PCR. Differentiated cells had increased levels of podocyte mRNA markers including WT1, PODXL, and SYNPO (FIG. 15Ei). Increased expression of the podocyte marker SYNPO in the differentiated podocytes following quantification of marker protein levels by Western blot analysis as well (FIG. 15Eii). Immunostaining indicated that iPSC-derived podocytes produced the actin-associated protein SYNPO in a filamentous pattern. As protocols starting with iPSCs tend to produce more immature cell types, it was not surprising that expression of SYNPO, which is specific to postmitotic differentiated podocytes, was lower (44.5%) than other markers that are less specific to mature podocytes (FIG. 15A). Scanning electron microscopy (SEM) analysis of the resulting podocytes revealed thin foot processes extending from cells (FIG. 15B). Transmission electron microscopy (TEM) analysis at higher magnification revealed tight junctions formed between podocytes (FIG. 15C).

Comparison of Different Methods of iPSC-Podocyte Differentiation

Next, four protocols for the differentiation of iPSC-derived podocytes derived from the DYR0100 iPSC line were carefully compared (FIG. 16). The differentiation efficiency of the accelerated protocol (Bejoy J, et al. STAR protocols. 2021 2 (4): 100898) was compared to Ciampi (Ciampi O, et al. Stem Cell Res. 2016 17 (1): 130-9), Rauch (Rauch C, et al. PloS one. 2018 13 (9): e0203869-e), and Musah (Musah S, et al. Nat Biomed Eng. 2017 1 (5): 1-12) protocols. Each protocol involves differentiation in different defined mediums, with major variation in the length of time required for each step and the composition of the various media (FIG. 16A). The protocol begins and ends with the same media as described in the Musah protocol, but was shortened by adding defined steps to reach the nephron progenitor cell type through modulation of the Wnt pathway. Whereas in the Musah protocol the concentration of the Wnt activator and glycogen-synthase kinase-3 (GSK-3) inhibitor CHIR99021 was held constant (Musah S, et al. Nat Biomed Eng. 2017 1 (5): 1-12), in this protocol CHIR99021 was increased from 3 μM to 8 μM for two days. Next, CHIR99021 was withdrawn to stop Wnt activation, similar to the 13-day Ciampi protocol (Ciampi O, et al. Stem Cell Res. 2016 17 (1): 130-9). To induce the nephron progenitor cell type, cells were cultured for two days in media including FGF9 stabilized by heparin without Wnt activation. This period required 4 days whereas the Musah protocol required 13 days (FIG. 16A). The other lengthy protocol, Rauch, does not rely on Wnt activation (Rauch C, et al. PloS one. 2018 13 (9): e0203869-e).

Immunofluorescence, flow cytometry analysis and Western blots were employed to compare the expression of podocyte-specific proteins (FIG. 4B-4D). Among the four protocols, Rauch had the lowest expression of each marker by every analysis technique (FIG. 16B-16E). Ciampi had low expression of SYNPO and NPHS1 by Western blot, but PODXL staining by Western, immunofluorescence and flow cytometry were high (FIG. 16B-16E). Compared to the Musah protocol, this protocol produced similar levels of podocyte marker PODXL and elevated levels of NPHS1 by immunofluorescence, comparable levels of MAFB and PODXL by flow cytometry analysis, and comparable levels of PODXL, SYNPO, and NPHS1 with higher levels of WT1 by Western blot (FIG. 4B-4D). All protocols had comparable numbers of cells that were MAFB+ or PODXL+ by flow cytometry analysis (FIG. 16Bi, 16Bii). iPSC-derived podocytes differentiated by the Ciampi and Rauch protocols have podocyte specification, but the expression levels of podocyte markers by Western blot were lower than the Musah or Bejoy protocols (FIG. 16D). Therefore, the shorter protocol produced iPSC-derived podocytes in similar numbers and with a maturity level that is equal to or better than established methods of podocyte differentiation.

iPSC-Derived Podocytes Integrated into Mouse Embryonic Kidney Organoids

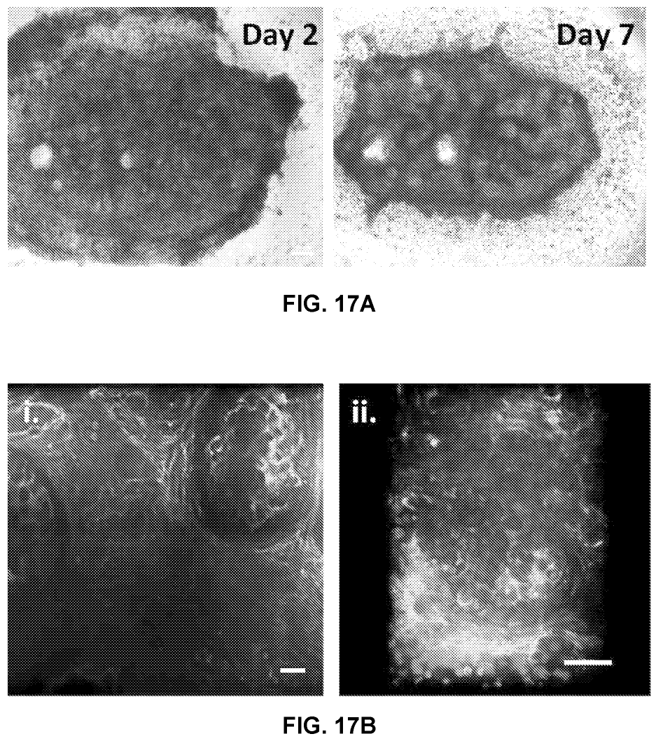

Ex vivo organoid culture is a widely accepted assay to verify the potential of cells to integrate into the expected cellular niche (Song B, et al. PloS one. 2012 7 (9): e46453; Hendry C E, et al. J Am Soc Nephrol. 2013 24 (9): 1424-34; Vanslambrouck J M, et al. Kidney Int. 2019 95 (5): 1153-66). Previous reports showed that nephron progenitor cells can integrate into the endogenous nephron progenitor field and can proliferate within the assay (Hendry C E, et al. J Am Soc Nephrol. 2013 24 (9): 1424-34; Vanslambrouck J M, et al. Kidney Int. 2019 95 (5): 1153-66). Therefore, the ability of iPSC-derived podocytes to integrate into the renal milieu was tested using cellular recombination (Song B, et al. PloS one. 2012 7 (9): e46453). Differentiated iPSC-podocytes were recombined with cells dissociated from mouse embryonic E12.5-E15.5 kidneys by centrifugation and the resulting pellet was explant cultured for 7 days (FIG. 17A). The aggregate was fixed on day 7. Both a human-specific MAFB antibody and a mouse-specific NPHS1 antibody were used to track the podocytes in the hybrid glomeruli. Immunostaining revealed close interaction of the MAFB+ iPSC-podocytes with mouse cells that were NPHS1+ within the glomeruli. MAFB+ human podocytes were predominantly concentrated on the periphery of aggregate recombinations (FIG. 17Bi, 17Bii).

iPSC-Derived Podocyte: Functional Validation and Modeling of Diabetic Kidney Disease

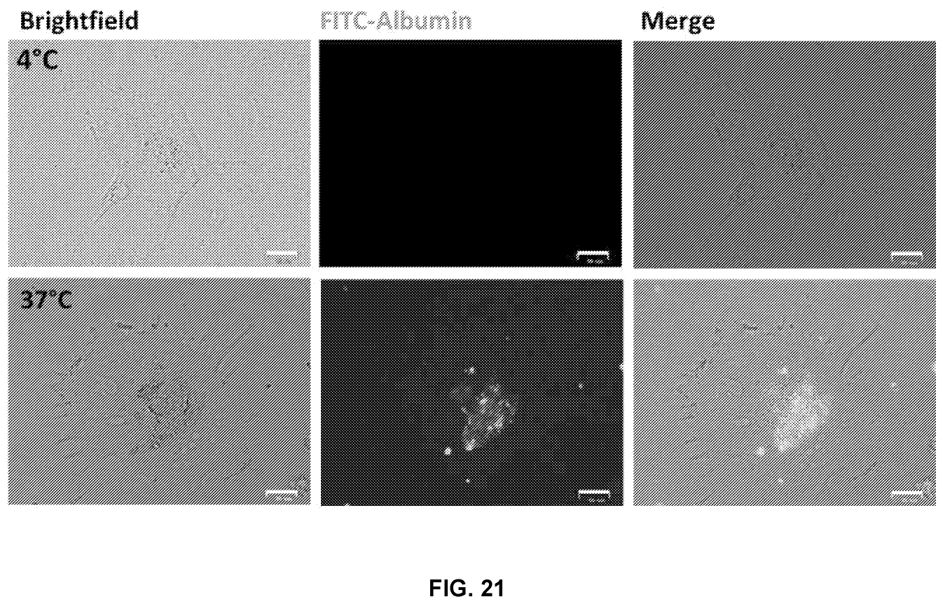

The endocytic absorption of albumin is an approximate measure of the functionality of podocytes. FITC-albumin uptake by iPSC-derived podocytes was measured by imaging on a fluorescence microscope. iPSC-derived podocytes cultured for 24 h at 37° C. had uptake of FITC-albumin into the cytoplasm, whereas podocytes cultured at 4° C. failed to endocytose albumin (Gheith O, et al. J Nephropharmacol. 2015 5 (1): 49-56) (FIG. 21). This temperature-dependent uptake is consistent with data from immortalized podocyte cell lines (Okamura K, et al. PLOS ONE. 2013 8 (1): e54817).

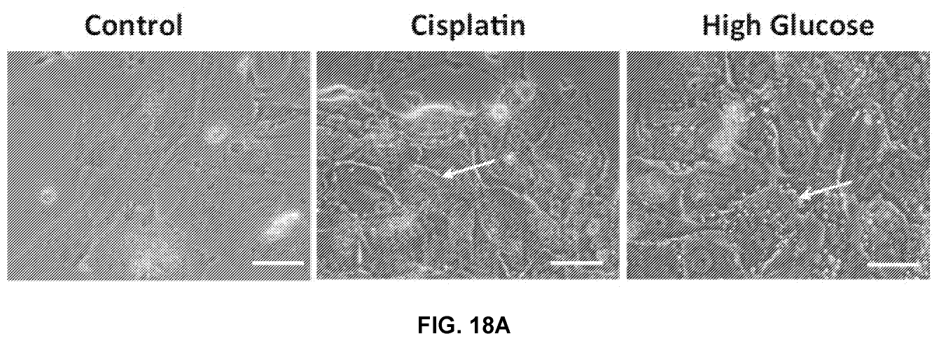

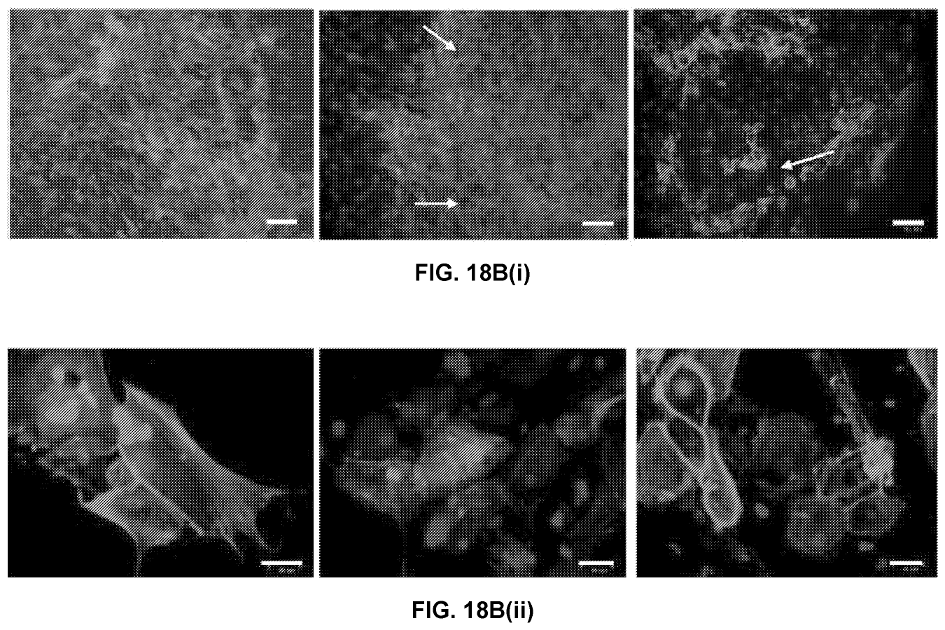

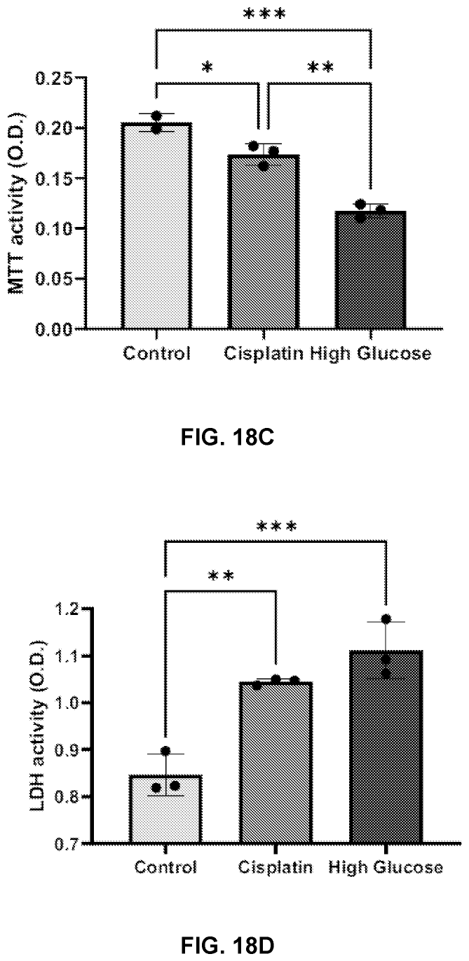

The differentiated podocytes were treated with 100 mM glucose for 48 h as a high glucose condition to model diabetic nephropathy. As a positive control for cell death, podocytes were treated with cisplatin. A nephrotoxic medication that damages MAFB+ cells (Digby J L M, et al. Am J Physiol Renal Physiol. 2020 318 (4): F971-F8). Brightfield images revealed that cells treated with either cisplatin or high glucose had lost their cellular integrity (FIG. 18). Compared to control cells, F-actin staining demonstrated cytoskeletal remodeling within treated cells (FIG. 18B). Higher magnification photos revealed disordered, entangled actin fibers in treated groups whereas control podocytes matched fasciculate models (FIG. 18B). Different biochemical assays were used to assess the damage caused by glucose to the podocytes. The high glucose-treated podocytes had lower viability than the untreated control group by MTT assay (FIG. 18C). To investigate the cytotoxicity, an LDH assay was performed, demonstrating that podocytes subjected to the high glucose condition had an increase in cytotoxicity (FIG. 18D). Together, these data indicate that iPSC-derived podocytes can be used to model glomerular disorders such as diabetic kidney disease.

### DISCUSSION

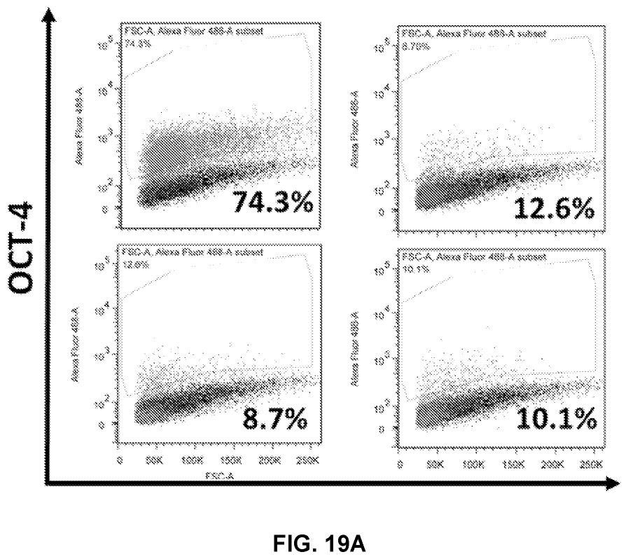

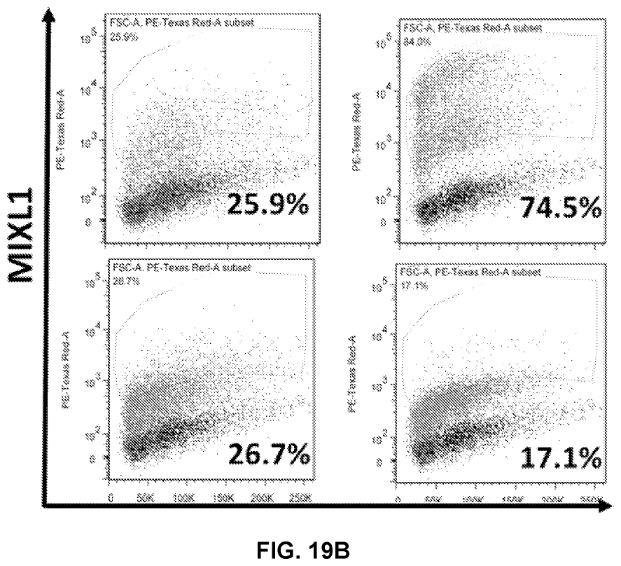

Although human primary podocytes can be isolated, dedifferentiation of cells in tissue culture causes cells to lose their podocyte identity over time (Ni L, et al. Nephrology 2012 17 (6): 525-31; Krtil J, et al. Kidney Blood Press Res. 2007 30 (3): 162-74). In addition, the limited availability of adult kidney samples and eventual senescence of primary cells limits the utility of primary CD133+/CD24+/PODXL+ cells isolated from adult human kidney (Ronconi E, et al. J Am Soc Nephrol. 2009 20 (2): 322-32). Although less cumbersome than primary cells to culture, conditionally immortalized podocytes also have limited utility for disease modeling due to poor expression of some podocyte markers (Chittiprol S, et al., Am J Physiol Renal Physiol. 2011 301 (3): F660-F71). Attempts to generate podocytes directly from iPSCs have resulted in immature podocytes with limited functionality (Ciampi O, et al. Stem Cell Res. 2016 17 (1): 130-9; Rauch C, et al. PloS one. 2018 13 (9): e0203869-e). The production of more functional iPSC-derived podocytes with higher levels of markers indicating maturity requires a longer culture time and more expensive medium components (Musah S, et al. Nat Biomed Eng. 2017 1 (5): 1-12). A four-step technique was devised to generate functional podocytes from iPSCs via nephron progenitors (Bejoy J, et al. STAR protocols. 2021 2 (4): 100898). Activation of activin signaling along with Wnt induced posterior primitive streak formation from iPSCs (Nostro M C, et al. Cell stem cell. 2008 2 (1): 60-71). Therefore, a combination of activin A with a lower concentration of the GSK-3 inhibitor/Wnt activator CHIR99021 was used to generate the posterior primitive streak (Bejoy J, et al. STAR protocols. 2021 2 (4): 100898). The primitive streak identity was confirmed by immunostaining for MIXL1 at day 2 (FIG. 14B and FIG. 19B). To direct cells to the intermediate mesoderm (Qian T, et al. Science Advances. 2017 3 (11): e1701679; Song B, et al. PloS one. 2012 7 (9): e46453; Ciampi O, et al. Stem Cell Res. 2016 17 (1): 130-9), high Wnt signaling activation was initiated from day 3 to day 5. For confirmation, there cells were stained for the intermediate mesoderm marker Paired Box 8 (PAX8; FIG. 14C). By withdrawing Wnt activation and treating with FGF9 plus heparin for stabilization, these intermediate mesoderm cells were subsequently differentiated into nephron progenitors and confirmed their identity with the markers SIX2 and CITED1 (FIG. 14D).

To generate podocytes from nephron progenitor cells, the epithelialization of these cells must be stimulated. One of the most essential growth factors in the differentiation of podocytes is RA. RA triggers stem cells to lose their self-renewing properties and differentiate. RA therefore modulates podocyte gene expression to encourage nephron progenitors to differentiate into podocytes. BMP7 is a member of the BMP family, which belongs to the TGF superfamily, and is found in a variety of organs including podocyte precursor cells (Dudley A T, et al. Genes & development. 1995 9 (22): 2795-807; Mitu G M, et al., Am J Physiol Renal Physiol. 2007 293 (5): F1641-F8). Activation of BMP7 signaling at some point in the protocol appears to be required for differentiation of podocytes from iPSCs (Ciampi O, et al. Stem Cell Res. 2016 17 (1): 130-9; Chugh S S. Transl Res. 2007 149 (5): 237-42; Li J, et al. Kidney International. 2007 72: S36-S42; Ni L, et al. Nephrology 2012 17 (6): 525-31). Another molecule important for podocyte survival is VEGF (Musah S, et al. Nat Biomed Eng. 2017 1 (5): 1-12). In the Musah protocol as well as in this study, a combination of BMP7, CHIR99021, activin A, RA, VEGF induced the differentiation of podocytes from nephron progenitors (Musah S, et al. Nat Biomed Eng. 2017 1 (5): 1-12)

The podocytes derived from this accelerated protocol had the typical arborized morphology, comprised of a main cell body with extending processess (FIG. 13B). The iPSC-derived podocytes expressed podocyte markers including SYNPO, NPHS1, MAFB, P cadherin and PODXL (FIG. 15A). Flow cytometry data demonstrated that the number of cells differentiated by each of the four protocols were comparable, between 58% and 75% (FIG. 16B-16D). Western blot analysis of the total protein levels showed highly variable expression between the four protocols, suggesting major differences in podocyte maturity among cells differentiated to a podocyte phenotype (FIG. 16D). The Rauch protocol had the lowest expression of protein markers, suggesting the poorest differentiation. Interestingly, withdrawal of Wnt activation was present in both the 12-day protocol and the Ciampi 13-day protocol whereas the longer protocols did not manipulate Wnt activation levels during the protocol; they were either activated with steady CHIR amounts (Musah) or not activated (Rauch). It is possible that oscillation of the Wnt pathway through modulation of the levels of Wnt activation may accelerate differentiation (Cantoria M J, et al. BioRxiv. 2022 2022.01.28.478206). Both the accelerated protocol and the Ciampi protocol produced iPSC-derived podocytes with increased WT1 expression. As WT1 interacts with the Wnt pathway, this may be a response to the CHIR withdrawal and reintroduction in both protocols. Future optimization of Wnt oscillation may be able to further accelerate iPSC-podocyte differentiation. The marker PODXL must be present in both mature and immature podocytes for normal glomerular function (Refaeli I, et al. Scientific Reports. 2020 10 (1): 9419). The protein levels of this marker were highest for the Musah protocol followed by this protocol, with low levels in iPSC-podocytes derived by the Ciampi and Rauch protocols. The mature podocyte marker SYNPO had high expression in cells derived by this accelerated protocol, with levels that were comparable to iPSC-podocytes derived via the Musah protocol. Both the protocol and the Musah protocol employed VEGF to mimic endothelial signalling and had the highest levels of podocyte marker proteins. Therefore, simulation of endothelial to podocyte crosstalk through the addition of VEGF may be one of the keys to induction of a more mature podocyte phenotype. NPHS1 levels were low but detectable, and the levels were comparable for all protocols (FIG. 16D).

The attraction of the differentiated iPSCs for their native niche was tested by integrating them into a developing kidney structure in a recombination assay with mouse embryonic kidney cells (FIG. 17A). This assay demonstrated podocyte identity via incorporation of MAFB+ iPSC-podocytes into NPHS1+ mouse glomeruli structures (FIG. 17B). The ability to internalize albumin is one of the important features of mature podocytes (Gianesello L, et al. Scientific Reports. 2017 7 (1): 13705). Therefore, iPSC-derived podocytes were incubated with FITC-albumin at various temperatures and showed temperature-dependent endocytosis of albumin (FIG. 21). With this functional validation in place, the utility of the iPSC-derived podocytes for disease modeling was investigated. Current human diabetic kidney disease models rely upon human renal biopsy samples to generate podocytes in vitro. In vitro diabetic nephropathy has been modeled by treating immortalized human podocytes with high glucose (Ling L, et al. Mol Med Rep. 2018 17 (4): 5642-51; Khazim K, et al. Am J Physiol Renal Physiol. 2013 305 (5): F691-F700). However, immortalization and lengthy periods in tissue culture change cell death pathways. In this study, treating iPSC-derived podocytes with high glucose damaged the integrity of actin filaments and changed the morphology of the podocytes (FIG. 18). Glucose treatment both increased podocyte cytotoxicity and decreased their viability. Immunofluorescence staining with phallodin showed extensive reorganization of F-actin along the cell periphery following the treatment with high glucose, suggesting severe podocyte dysfunction (FIG. 18).

### CONCLUSIONS

The disclosed accelerated method was able to produce podocytes that were comparable to existing methods by multiple markers. A faster and lower-cost method to generate podocytes from iPSCs was found that was based on mimicking the developmental stages of the embryonic kidney. The immunostaining of the podocytes showed positive staining for podocyte markers including PODXL, NPHS1, SYNPO, and MAFB. Functionality was verified by endocytosis of FITC-albumin. Furthermore, a recombination assay of the iPSC-derived podocytes with renal cells from E12.5 mouse embryos showed integration of iPSC-derived MAFB+ podocytes into NPHS1+ mouse glomeruli structures. Treatment of the derived podocytes with high glucose resulted in both actin rearrangement and cell death, suggesting the efficacy of these cells in modeling diabetic nephropathy. The understanding of both genetic and environmental podocyte diseases relies upon human tissue culture models that are both practical and faithful to phenotypes found in vivo. Using this method, the ability to generate patient-specific podocytes will provide a new resource, with future applications in drug screening and genome editing.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A method for producing podocytes, the method comprising:
(a) culturing the human pluripotent stem cells in a primitive streak induction medium comprising Activin A and/or Noggin and a glycogen synthase kinase 3 (GSK3) inhibitor and/or Wnt3a to produce posterior primitive streak cells;
(b) culturing the posterior primitive streak cells in an intermediate mesoderm induction medium comprising a GSK3 inhibitor to produce intermediate mesodermal cells;
(c) culturing the intermediate mesodermal cells of claim 1 in a nephron progenitor induction medium comprising fibroblast growth factor 9 (FGF9) and heparin to produce nephron progenitor cells; and
(d) culturing the nephron progenitor cells of claim 2 in a podocyte induction medium comprising bone morphogenetic protein 7 (BMP7), vascular endothelial growth factor (VEGF), a GSK3 inhibitor, retinoic acid, and Activin A to produce podocytes.

2-3. (canceled)

4. The method of claim 1, wherein the GSK3 inhibitor comprises CHIR99021.

5. The method of claim 1, wherein the primitive streak induction medium further comprises a Rho-associated, coiled-coil containing protein kinase (ROCK) inhibitor.

6. The method of claim 5, wherein the ROCK inhibitor comprises Y-27632.

7. The method of claim 1, wherein the primitive streak induction medium comprises 5-10 μM Y-27632, 10-100 ng/mL Activin A, and 1-3 μM CHIR99021,
wherein the intermediate mesoderm induction medium comprises 6-10 μM CHIR99021,
wherein the nephron progenitor induction medium comprise 50-200 ng/μl FGF9, and 1-10 μg/mL heparin, and
wherein the podocyte induction medium comprises 50-100 ng/ml BMP-7, 25-50 ng/mL VEGF, 1-3 μM CHIR99021, 0.1-1.0 μM all-trans retinoic acid, and 10-100 ng/ml Activin A.

8-10. (canceled)

11. The method of claim 1, wherein the human pluripotent stem cells, posterior primitive streak cells, intermediate mesodermal cells, nephron progenitor cells, or any combination thereof are cultured on a feeder free culture plate.

12. The method of claim 11, wherein the culture plates are laminin coated.

13. The method of claim 1, further comprising:
assaying the posterior primitive streak cells produced in step (a) for MIXL1 expression,
assaying the intermediate mesodermal cells produced in step (b) for paired gene box 8 (PAX8) and/or GATA3 expression,
assaying the nephron progenitor cells produced in step (c) for SIX2 and/or CITED1 expression, and
assaying the podocytes produced in step (d) for synaptopodin (SYNPO), podocalyxin (PODXL), MAF BZIP transcription factor (MAFB), NPHS1 Adhesion Molecule (Nephrin), or any combination thereof.

14-16. (canceled)

17. The method of claim 1,
wherein step (a) comprises culturing the human pluripotent stem cells for 2-4 days to produce the posterior primitive streak cells,
wherein step (b) comprises culturing the posterior primitive streak cells for 2-3 days to produce the intermediate mesodermal cells,
wherein step (c) comprises culturing the intermediate mesodermal cells for 2-3 days to produce the nephron progenitor cells, and
wherein step (d) comprises culturing the nephron progenitor cells for 5-7 days to produce the podocytes.

18-20. (canceled)

21. The method of claim 1, wherein the podocytes are produced from the human pluripotent stem cells within 12 days.

22. The method of claim 1, wherein the human pluripotent stem cells are induced pluripotent stem cells (iPSCs) or human embryonic stem cells.

23. The method of claim 22, wherein the iPSCs are genetically modified to model genetic kidney disease.

24-25. (canceled)

26. A composition comprising podocytes produced by the method of claim 1.

27. A method, comprising culturing the podocytes of claim 26 in a cell culture medium.

28-31. (canceled)

32. A method for treating a kidney disease in a subject, comprising administering to the subject a therapeutically effective amount of the podocytes of claim 26.

33-34. (canceled)


SEQUENCE LISTING (8 sequences)

SEQ ID NO: 1 - DNA, 20 bp, synthetic construct
  source 1..20: mol_type = other DNA
gatgtcaaccaaaaccttgc

SEQ ID NO: 2 - DNA, 20 bp, synthetic construct
  source 1..20: mol_type = other DNA
gtgccattagatgggagttg

SEQ ID NO: 3 - DNA, 20 bp, synthetic construct
  source 1..20: mol_type = other DNA
aggcttgagtgaggtgtttg

SEQ ID NO: 4 - DNA, 20 bp, synthetic construct
  source 1..20: mol_type = other DNA
agcctttgattgatttgcag

SEQ ID NO: 5 - DNA, 20 bp, synthetic construct
  source 1..20: mol_type = other DNA
tcatcactgggagtgtcctt

SEQ ID NO: 6 - DNA, 20 bp, synthetic construct
  source 1..20: mol_type = other DNA
tggatttcctcacccagtaa

SEQ ID NO: 7 - DNA, 21 bp, synthetic construct
  source 1..21: mol_type = other DNA
ctctctgctcctcctgttcga

SEQ ID NO: 8 - DNA, 18 bp, synthetic construct
  source 1..18: mol_type = other DNA
tgagcgatgtggctcggc